(19) United States
(12) Patent Application Publication
(10) Pub. No.: US 2011/0183928 A1
(43) Pub. Date: Jul. 28, 2011

(54) 3-Cyanoalkyl- and 3-hydroxyalkylindoles and use thereof

(75) Inventors: Kai Thede, Berlin (DE); Elisabeth Woltering, Hilden (DE); Peter Kolkhof, Wuppertal (DE); Carsten Schmeck, Mulheim (DE); Elisabeth Pook, Wuppertal (DE); Alexander Hillisch, Solingen (DE); Lars Bärfacker, Oberhausen (DE); Klemens Lustig, Wuppertal (DE); Dieter Lang, Velbert (DE); Martin Radtke, Erkrath (DE); Rolf Grosser, Leverkusen (DE); Astrid Brüns, Wuppertal (DE); Michael Gerisch, Wuppertal (DE)

(73) Assignee: BAYER SCHERING PHARMA AKTIENGESELLSCHAFT, Berlin (DE)

(21) Appl. No.: 13/001,095

(22) PCT Filed: Jun. 18, 2009
(86) PCT No.: PCT/EP2009/004392

§ 371 Date: Apr. 6, 2011

(30) Foreign Application Priority Data
Jun. 25, 2008 (DE) 10 2008 030 206.6

(51) Int. Cl. A61K 31/404; C07D 209/12; C07D 209/18; C07D 471/04; C07D 409/06; A61K 31/437; A61K 31/616; A61K 31/7048; A61P 9/12; A61P 9/00; A61P 13/12; A61P 1/16; A61P 5/40; A61P 9/04

(52) U.S. Cl. 514/26; 548/509; 548/505; 546/113; 548/468; 548/454; 514/415; 514/300; 514/414; 514/161

(57) ABSTRACT

The present application relates to novel 3-cyanoalkyl- and 3-hydroxyalkyl-substituted indole derivatives, to processes for preparation thereof, to the use thereof alone or in combinations for treatment and/or prevention of diseases, and to the use thereof for production of medicaments for treatment and/or prevention of diseases, especially for treatment and/or prevention of cardiovascular diseases.

The present application relates to novel 3-cyanoalkyl- and 3-hydroxyalkyl-substituted indole derivatives, to processes for preparation thereof, to the use thereof alone or in combinations for treatment and/or prevention of diseases, and to the use thereof for production of medicaments for treatment and/or prevention of diseases, especially for treatment and/or prevention of cardiovascular diseases.

Aldosterone plays a key role in maintaining liquid and electrolyte homeostasis, by promoting sodium retention and potassium secretion in the epithelium of the distal nephron, which contributes to keeping the extracellular volume constant, and hence to regulation of blood pressure. In addition, aldosterone displays direct effects on the structure and function of the cardiac and vascular system, though the underlying mechanisms are yet to be explained exhaustively [R. E. Booth, J. P. Johnson, J. D. Stockand, Adv. Physiol. Educ. 26 (1), 8-20 (2002)].

Aldosterone is a steroid hormone which is formed in the adrenal cortex. Production thereof is regulated indirectly, very substantially as a function of renal blood flow. Any decrease in renal blood flow leads to release in the kidney of the enzyme renin into the bloodstream. This in turn activates the formation of angiotensin II, which firstly has a constricting effect on the arterial blood vessels, but secondly also stimulates the formation of aldosterone in the adrenal cortex. The kidney thus functions as a sensor of blood pressure and hence indirectly of volume in the bloodstream, and counteracts critical losses of volume via the renin-angiotensin-aldosterone system, firstly by increasing the blood pressure (angiotensin II effect), and secondly by rebalancing the filling state of the vascular system by enhanced reabsorption of sodium and water in the kidney (aldosterone effect).

This regulation system can be pathologically impaired in various ways. For instance, a chronic reduction in renal blood flow (for example owing to heart failure and the congestion of blood in the venous system caused thereby) leads to a chronically excessive release of aldosterone. This in turn results in an expansion in the blood volume, thereby aggravating the weakness of the heart due to an excessive supply of volume to the heart. The results may be congestion of blood in the lungs causing shortness of breath and formation of edema in the extremities, and also ascites and pleural effusions; renal blood flow falls further. Moreover, the overenhanced aldosterone effect leads to a reduction in the potassium concentration in the blood and in the extracellular fluid. In heart muscles with existing damage in any case, potassium concentrations below a critical minimum level can trigger cardiac arrythmias with fatal consequences. This is likely to be one of the main causes of sudden cardiac death, which is a frequent occurence in patients with heart failure.

In addition, aldosterone is also thought to be responsible for a series of myocardial remodeling processes typically observed in patients with heart failure. Thus, hyperaldosteronism is a crucial component in the pathogenesis and prognosis of heart failure, the original trigger of which may be different kinds of damage, for example myocardial infarction, myocardial inflammation or high blood pressure. This assumption is reinforced by the fact that overall mortality was lowered significantly in extensive clinical studies in patient groups with chronic heart failure or after acute myocardial infarction by use of aldosterone antagonists [B. Pitt, F. Zannad, W. J. Remme et al., N. Engl. J. Med. 341, 709-717 (1999); B. Pitt, W. Remme, F. Zannad et al., N. Engl. J. Med. 348, 1309-1321 (2003)]. One way of achieving this was by lowering the incidence of sudden cardiac death.

According to recent studies, a not inconsiderable number of patients suffering from essential hypertension are also found to have what is known as a normokalemic variant of primary hyperaldosteronism [prevalence up to 11% of all hypertensives: L. Seiler and M. Reincke, Der Aldosteron-Renin-Quotient bei sekundärer Hypertonia, Herz 28, 686-691 (2003)]. The best diagnosis method used in the case of normokalemic hyperaldosteronism is the aldosterone/renin ratio of the corresponding plasma concentrations, such that even relative aldosterone increases in relation to the renin plasma concentration become amenable to diagnosis and ultimately treatment. Therefore, hyperaldosteronism diagnosed in combination with essential hypertension is a starting point for causal and prophylactically viable treatment.

Pathogenic states much less commonly encountered than the forms of hyperaldosteronism detailed above are those in which either the impairment is to be found in the hormone-producing cells of the adrenal gland itself, or the number or mass thereof is increased as a result of hyperplasia or proliferation. Adenomas or diffuse hyperplasias of the adrenal cortex are the most common cause of primary aldosteronism, also referred to as Conn's syndrome, the key symptoms of which are hypertension and hypokalemic alkalosis. Here too, in addition to the surgical removal of the diseased tissue, the emphasis is on medical treatment with aldosterone antagonists [H. A. Kühn and J. Schirmeister (eds.), Innere Medizin, 4th ed., Springer Verlag, Berlin, 1982].

Another pathogenic state typically associated with an increase in the aldosterone concentration in the plasma is advanced cirrhosis of the liver. The main cause of the aldosterone increase here lies in the limited degradation of the aldosterone owing to impaired liver function. Volume overload, edema and hypokalemia are the typical consequences, which can be alleviated successfully in clinical practice by aldosterone antagonists.

The effects of aldosterone are mediated via the mineralocorticoid receptor localized intracellularly in the target cells. The aldosterone antagonists available to date, like aldosterone itself, have a steroid-based structure. The employability of such steroidal antagonists is restricted by their interactions with the receptors of other steroid hormones, some of which lead to considerable side effects such as gynecomastia and impotence, and to stoppage of the treatment [M. A. Zaman, S. Oparil, D. A. Calhoun, Nature Rev. Drug Disc. 1, 621-636 (2002)].

The identification of potent, nonsteroidal antagonists which are selective for the mineralocorticoid receptor opens up the possibility of avoiding this profile of side effects and thus achieving a distinct therapeutic benefit [cf. M. J. Meyers and X. Hu, Expert Opin. Ther. Patents 17 (1), 17-23 (2007)].

It is therefore an object of the present invention to provide novel compounds which act as potent and selective mineralocorticoid receptor antagonists and can thus be used for the treatment of diseases, and especially of cardiovascular diseases.

WO 2004/067529, WO 2005/092854 and M. G. Bell, J. Med. Chem. 2007, 50 (26), 6443-6445 describe various 3-substituted indole derivatives as modulators of steroid hormone receptors. Indol-3-yl(phenyl)acetic acid derivatives as endothelin receptor antagonists are disclosed in WO 97/43260, and α-amino(indol-3-yl)acetic acid derivatives with antidiabetic action are disclosed in WO 90/05721. WO 2007/062994 and WO 2005/118539 claim 3-(3-amino-1-arylpropyl)indoles for treatment of depression and states of anxiety. 3-(Indol-3-yl)-3-phenylpropionitrile derivatives are disclosed in U.S. Pat. No. 2,752,358, U.S. Pat. No. 2,765,320 and U.S. Pat. No. 2,778,819 inter alia. The preparation of 2-unsubstituted indoles is disclosed in WO 98/06725 and U.S. Pat. No. 5,808,064. The preparation of 2-(indol-3-yl)-2-phenylethanol derivatives is reported inter alia in M. L. Kantam et al., Tetrahedron Lett. 47 (35), 6213-6216 (2006). EP 0 778 277-A1 discloses various azabicyclic compounds as CRF antagonists. WO 2007/040166 claims fused pyrrole derivatives as glucocorticoid receptor modulators with antiinflammatory and antidiabetic action. WO 2007/070892 describes substituted indoles for treatment of anxiety, pain and cognitive disorders.

The present invention provides compounds of the general formula (I)

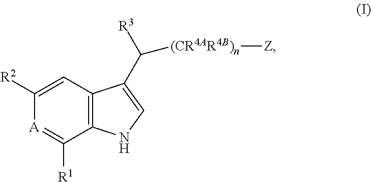

in which
A is C—R⁵ or N
   where
   R⁵ is hydrogen, fluorine, chlorine or (C₁-C₄)-alkyl,
R¹ is halogen, cyano, nitro, (C₁-C₆)-alkyl, (C₁-C₆)-alkoxy, amino, mono-(C₁-C₆)-alkylamino, di-(C₁-C₆)-alkylamino or a group of the formula —(CH₂)ₚ—NR⁶—SO₂—R⁷,
   where (C₁-C₆)-alkyl and (C₁-C₆)-alkoxy may each be substituted by 1 to 3 fluorine substituents,
   where (C₁-C₆)-alkyl and (C₁-C₆)-alkoxy may each be substituted by one substituent selected from the group of hydroxyl and (C₁-C₄)-alkoxy,
   and where
   p is 0, 1 or 2,
   R⁶ is hydrogen or (C₁-C₄)-alkyl,
   and
   R⁷ is (C₁-C₆)-alkyl, (C₃-C₇)-cycloalkyl, phenyl, benzyl or 5- or 6-membered heteroaryl,
      in which phenyl, benzyl and 5- or 6-membered heteroaryl may each be substituted by 1 to 3 substituents selected independently from the group of halogen, cyano, nitro, (C₁-C₄)-alkyl, trifluoromethyl, hydroxyl, (C₁-C₄)-alkoxy, trifluoromethoxy and amino,
R² is hydrogen, fluorine, chlorine or (C₁-C₄)-alkyl,
R³ is phenyl or naphthyl,
   where phenyl and naphthyl may each be substituted by 1 to 3 substituents selected independently from the group of halogen, cyano, nitro, (C₁-C₄)-alkyl, trifluoromethyl, (C₁-C₄)-alkoxy, trifluoromethoxy, mono-(C₁-C₄)-alkylamino, di-(C₁-C₄)-alkylamino, aminocarbonyl, mono-(C₁-C₄)-alkylaminocarbonyl and di-(C₁-C₄)-alkylaminocarbonyl,
n is 2 or 3,
R^{4A} is hydrogen, fluorine or (C₁-C₄)-alkyl,
R^{4B} is hydrogen, fluorine or (C₁-C₄)-alkyl,
and
Z is hydroxyl or cyano,
   and the salts, solvates and solvates of the salts thereof.

Inventive compounds are the compounds of the formula (I) and the salts, solvates and solvates of the salts thereof, the compounds, encompassed by formula (I), of the formulae specified hereinafter and the salts, solvates and solvates of the salts thereof, and the compounds encompassed by formula (I) and specified hereinafter as working examples and the salts, solvates and solvates of the salts thereof, to the extent that the compounds encompassed by formula (I) and specified hereinafter are not already salts, solvates and solvates of the salts.

Depending on their structure, the inventive compounds may exist in stereoisomeric forms (enantiomers, diastereomers). The invention therefore encompasses the enantiomers or diastereomers and the respective mixtures thereof. The stereoisomerically homogeneous constituents can be isolated from such mixtures of enantiomers and/or diastereomers in a known manner.

Where the inventive compounds can occur in tautomeric forms, the present invention encompasses all the tautomeric forms.

In the context of the present invention, preferred salts are physiologically acceptable salts of the inventive compounds. Also encompassed are salts which are not themselves suitable for pharmaceutical applications but can be used, for example, for isolation or purification of the inventive compounds.

Physiologically acceptable salts of the inventive compounds include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates refer to those forms of the inventive compounds which, in the solid or liquid state, form a complex by coordination with solvent molecules. Hydrates are a specific form of the solvates in which the coordination is with water. Hydrates are preferred solvates in the context of the present invention.

Moreover, the present invention also encompasses prodrugs of the inventive compounds. The term “prodrugs” includes compounds which may themselves be biologically active or inactive but are converted to inventive compounds while resident in the body (for example metabolically or hydrolytically).

In the context of the present invention, unless specified otherwise, the substituents are defined as follows:

Alkyl in the context of the invention is a linear or branched alkyl radical having 1 to 6 or 1 to 4 carbon atoms. Preference is given to a linear or branched alkyl radical having 1 to 4 carbon atoms. Preferred examples include: methyl, ethyl, n-propyl, isopropyl, n-butyl, isobutyl, sec-butyl, tert-butyl, 1-ethylpropyl, n-pentyl and n-hexyl.

Cycloalkyl in the context of the invention is a monocyclic saturated carbocycle having 3 to 7 or 3 to 6 ring carbon atoms. Preferred examples include: cyclopropyl, cyclobutyl, cyclopentyl, cyclohexyl and cycloheptyl.

Alkoxy in the context of the invention is a linear or branched alkoxy radical having 1 to 6 or 1 to 4 carbon atoms. Preference is given to a linear or branched alkoxy radical having 1 to 4 carbon atoms. Preferred examples include: methoxy, ethoxy, n-propoxy, isopropoxy, n-butoxy, tert-butoxy, n-pentoxy and n-hexoxy.

Monoalkylamino in the context of the invention is an amino group having a linear or branched alkyl substituent which has 1 to 6 or 1 to 4 carbon atoms. Preference is given to a linear or branched monoalkylamino radical having 1 to 4 carbon atoms. Preferred examples include: methylamino, ethylamino, n-propylamino, isopropylamino, n-butylamino, tert-butylamino, n-pentylamino and n-hexylamino.

Dialkylamino in the context of the invention is an amino group having two identical or different, linear or branched alkyl substituents, each of which has 1 to 6 or 1 to 4 carbon atoms. Preference is given to linear or branched dialkylamino radicals each having 1 to 4 carbon atoms. Preferred examples include: N,N-dimethylamino, N,N-diethylamino, N-ethyl-N-methylamino, N-methyl-N-n-propylamino, N-isopropyl-N-n-propylamino, N,N-diisopropylamino, N-n-butyl-N-methylamino, N-tert-butyl-N-methylamino, N-ethyl-N-n-pentylamino and N-n-hexyl-N-methylamino.

Monoalkylaminocarbonyl in the context of the invention is an amino group which is attached via a carbonyl group and has a linear or branched alkyl substituent having 1 to 4 carbon atoms. Preferred examples include: methylaminocarbonyl, ethylaminocarbonyl, n-propylaminocarbonyl, isopropyl-aminocarbonyl, n-butylaminocarbonyl, tert-butylaminocarbonyl, n-pentylaminocarbonyl and n-hexylaminocarbonyl.

Dialkylaminocarbonyl in the context of the invention is an amino group which is attached via a carbonyl group and has two identical or different, linear or branched alkyl substituents each having 1 to 4 carbon atoms. Preferred examples include:

N,N-dimethylaminocarbonyl, N,N-diethylaminocarbonyl, N-ethyl-N-methylaminocarbonyl, N-methyl-N-n-propylaminocarbonyl, N-n-butyl-N-methylaminocarbonyl, N-tert-butyl-N-methylaminocarbonyl, N-n-pentyl-N-methylaminocarbonyl and N-n-hexyl-N-methylaminocarbonyl.

Heteroaryl in the context of the invention is a monocyclic aromatic heterocycle (heteroaromatic) which has a total of 5 or 6 ring atoms, contains up to three identical or different ring heteroatoms from the group of N, O and/or S and is attached via a ring carbon atom or optionally via a ring nitrogen atom. Preferred examples include: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl, triazinyl. Preference is given to monocyclic 5- or 6-membered heteroaryl radicals having up to two ring heteroatoms from the group of N, O and/or S, for example furyl, thienyl, thiazolyl, oxazolyl, isothiazolyl, isoxazolyl, pyrazolyl, imidazolyl, pyridyl, pyrimidinyl, pyridazinyl, pyrazinyl.

Halogen in the context of the invention includes fluorine, chlorine, bromine and iodine. Preference is given to chlorine or fluorine.

If radicals in the inventive compounds are substituted, the radicals may be mono- or polysubstituted, unless specified otherwise. In the context of the present invention, all radicals which occur more than once are defined independently of one another. Substitution by one or two identical or different substituents is preferred. Very particular preference is given to substitution by one substituent.

Preference is given to compounds of the formula (I) in which

### A is C—R⁵

where
R⁵ is hydrogen,
R¹ is chlorine, bromine, cyano, nitro, (C₁-C₄)-alkyl, (C₁-C₄)-alkoxy, amino, mono-(C₁-C₄)-alkylamino, di-(C₁-C₄)-alkylamino or a group of the formula —(CH₂)ₚ—NR⁶—SO₂—R⁷,
   where (C₁-C₄)-alkyl and (C₁-C₄)-alkoxy may each be substituted by 1 to 3 fluorine substituents,
   where (C₁-C₄)-alkyl and (C₁-C₄)-alkoxy may each be substituted by a substituent selected from the group of hydroxyl and (C₁-C₄)-alkoxy,
   and where
   p is 0 or 1,
   R⁶ is hydrogen or methyl,
   and
   R⁷ is (C₁-C₆)-alkyl, (C₃-C₆)-cycloalkyl, phenyl, benzyl or 5- or 6-membered heteroaryl,
      in which phenyl, benzyl and 5- or 6-membered heteroaryl may each be substituted by 1 or 2 substituents selected independently from the group of fluorine, chlorine, bromine, cyano, nitro, (C₁-C₄)-alkyl, trifluoromethyl, hydroxyl, (C₁-C₄)-alkoxy, trifluoromethoxy and amino,
R² is hydrogen, fluorine or methyl,
R³ is phenyl or naphthyl,
   where phenyl and naphthyl may each be substituted by 1 or 2 substituents selected independently from the group of fluorine, chlorine, bromine, cyano, nitro, (C₁-C₄)-alkyl, trifluoromethyl, (C₁-C₄)-alkoxy and trifluoromethoxy,
n is 2 or 3,
R^{4A} is hydrogen, fluorine or methyl,
R^{4B} is hydrogen, fluorine or methyl,
and
Z is hydroxyl or cyano,
   and the salts, solvates and solvates of the salts thereof.

Particular preference is given to compounds of the formula (I) in which
A is C—R⁵
   where
   R⁵ is hydrogen,
R¹ is bromine, cyano, methyl, ethyl, trifluoromethyl or a group of the formula —(CH₂)ₚ—NR⁶—SO₂—R⁷,
   and where
   p is 0,
   R⁶ is hydrogen,
   and
   R⁷ is methyl or ethyl,
R² is hydrogen or fluorine,
R³ is phenyl or naphthyl,
   where phenyl may be substituted by 1 or 2 substituents selected independently from the group of fluorine, chlorine, methyl and trifluoromethyl,
n is 2 or 3,
R^{4A} is hydrogen,
R^{4B} is hydrogen,
and
Z is hydroxyl or cyano,
   and the salts, solvates and solvates of the salts thereof.

The individual radical definitions specified in the respective combinations or preferred combinations of radicals are, independently of the respective combinations of the radicals specified, also replaced as desired by radical definitions of other combinations.

Very particular preference is given to combinations of two or more of the preferred ranges mentioned above.

The invention further provides a process for preparing the inventive compounds of the formula (I), characterized in that
[A] first an indole derivative of the formula (II)

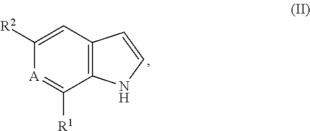

in which A, R¹ and R² are each as defined above,
in an inert solvent, optionally in the presence of an acid and/or base, is condensed with a benzaldehyde of the formula (III)

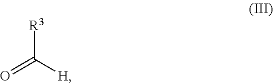

in which R³ is as defined above,
and a malonic ester of the formula (IV)

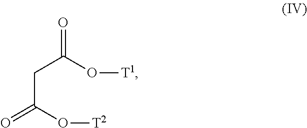

in which
T¹ and T² are the same or different and are each (C₁-C₄)-alkyl, or both together form a >C(CH₃)₂ bridge,
to give a compound of the formula (V)

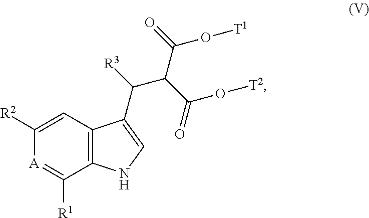

in which A, R¹, R², R³, T¹ and T² are each as defined above,
then the diester is cleaved with decarboxylation to give a compound of the formula (VI)

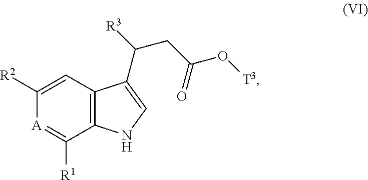

in which A, R¹, R² and R³ are each as defined above
and
T³ is hydrogen or (C₁-C₄)-alkyl,
and the latter compound is then converted in an inert solvent, using a suitable reducing agent, for example lithium aluminum hydride, to the inventive compound of the formula (I-1)

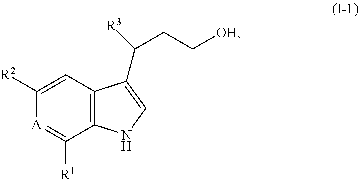

in which A, R¹, R² and R³ are each as defined above,
[B] the compound of the formula (I-1) is in turn reacted by standard methods, via a compound of the formula (VII)

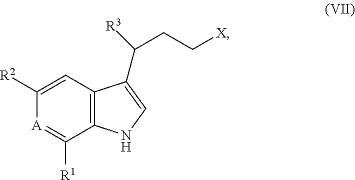

in which A, R¹, R² and R³ are each as defined above
and
X is a suitable leaving group, for example halogen, mesylate, tosylate or triflate,
and subsequent substitution reaction with an alkali metal cyanide to give the inventive compound of the formula (I-2)

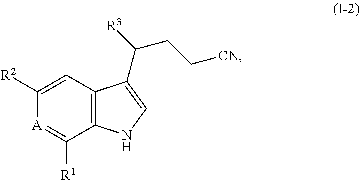

in which A, R¹, R² and R³ are each as defined above,
[C] the compound of the formula (I-2) is in turn first hydrolyzed to the carboxylic acid of the formula (VIII)

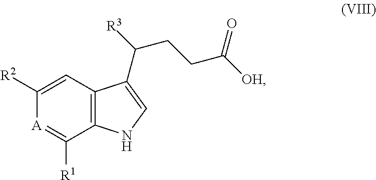

in which A, R¹, R² and R³ are each as defined above,
and the latter compound is then converted in an inert solvent, using a suitable reducing agent, for example lithium aluminum hydride, to the inventive compound of the formula (I-3)

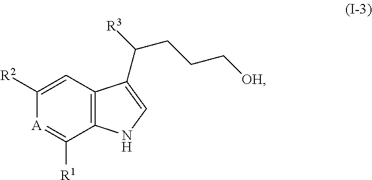

in which A, R¹, R² and R³ are each as defined above,
and
[D] the compound of the formula (I-3) is in turn reacted by standard methods, via a compound of the formula (IX)

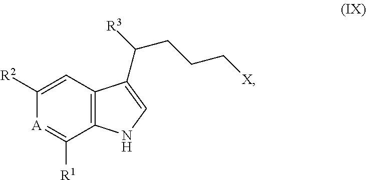

in which A, R¹, R² and R³ are each as defined above
and
X is a suitable leaving group, for example halogen, mesylate, tosylate or triflate, and subsequent substitution reaction with an alkali metal cyanide to give the inventive compound of the formula (I-4)

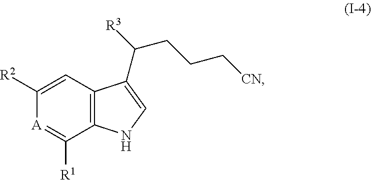

in which A, R¹, R² and R³ are each as defined above,
   and the resulting compounds of the formula (I-1), (I-2), (I-3) or (I-4) are optionally separated by methods known to those skilled in the art into the enantiomers and/or diastereomers thereof and/or converted using the appropriate (i) solvents and/or (ii) bases or acids to the solvates, salts and/or solvates of the salts thereof.

Further inventive compounds can optionally also be prepared by conversions of functional groups of individual substituents, especially those listed for R¹ and R³, proceeding from compounds of the formula (I) obtained by above processes. These conversions are performed by customary methods known to those skilled in the art and include, for example, reactions such as nucleophilic, electrophilic or transition metal-catalyzed substitution reactions, oxidation, reduction, hydrogenation, alkylation, acylation, amination, esterification, ester cleavage, etherification, ether cleavage, formation of carbonamides and sulfonamides, and the introduction and removal of temporary protecting groups [cf. also synthesis schemes 2-7 below].

Inventive compounds of the formula (I) in which individual R^{4A} and/or R^{4B} radicals are fluorine or (C₁-C₄)-alkyl can be prepared by known methods for fluorination or alkylation of carbonyl compounds proceeeding from the above-described compounds of the formulae (VI), (VIII), (1-2) or (1-4) [cf., for example, Z. Xu et al., J. Fluorine Chem. 58 (1), 71-79 (1992); A. Malabarba et al., Farmaco Ed. Sci. 39 (12), 1050-1060 (1984)].

The process step (II)+(III)+(IV)→(V) can be performed in one stage as a 3-component reaction, or else in two stages, by first condensing the benzaldehyde of the formula (III) with the malonic ester of the formula (IV) by standard methods to give a benzylidene compound of the formula (X)

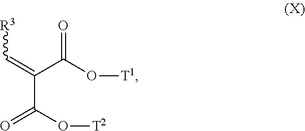

in which R³, T¹ and T² are each as defined above,
and then reacting the latter compound with the indole of the formula (II) in a separate reaction step.

In the one-stage reaction regime (II)+(III)+(IV)→(V), the malonic ester component (IV) used is preferably Meldrum's acid (cyclic isopropylidene malonate). The resulting product of the formula (Va)

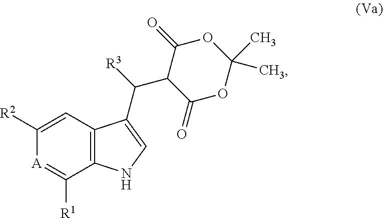

in which A, R¹, R² and R³ are each as defined above,
is subsequently converted by solvolysis with methanol or ethanol in the presence of pyridine and copper powder to an ester of the formula (VI) [T³=methyl or ethyl; cf. Y. Oikawa et al., Tetrahedron Lett., 1759-1762 (1978)].

The one-stage process variant (II)+(III)+(IV)→(V) and—in the case of a two-stage reaction regime—the condensation (III)+(IV)→(X) are preferably performed in the presence of an acid/base catalyst, for example D,L-proline or piperidinium acetate. The reaction (X)+(II)→(V) can in some cases be accomplished advantageously with the aid of an amine base such as triethylamine, or of a Lewis acid such as copper(II) trifluoromethanesulfonate or ytterbium trifluoromethanesulfonate.

Suitable solvents for process steps (II)+(III)+(IV)→(V) and (X)+(II)→(V) are all organic solvents which are inert under the reaction conditions. These include acyclic and cyclic ethers such as diethyl ether, methyl tert-butyl ether, 1,2-dimethoxyethane, tetrahydrofuran and dioxane, hydrocarbons such as benzene, toluene, xylene, hexane and cyclohexane, chlorinated hydrocarbons such as dichloromethane, trichloromethane and chlorobenzene, or dipolar aprotic solvents such as dimethylformamide (DMF), dimethyl sulfoxide (DMSO), N-methylpyrrolidinone (NMP) and acetonitrile. It is equally possible to use mixtures of the solvents mentioned. Preference is given to using acetonitrile.

The reactions are effected generally within a temperature range from 0° C. to +120° C., preferably at 0° C. to +60° C. The reactions can be performed at standard, elevated or reduced pressure (for example in the range from 0.5 to 5 bar). The working pressure is generally atmospheric pressure.

A suitable reducing agent in process steps (VI) (I-1) and (VIII)→(I-3) is especially lithium aluminum hydride or lithium borohydride. In the case of the carboxylic acids (VIII) and (VI) [T³=H], it is alternatively also possible to use diborane or borane complexes. The reactions are preferably performed in an ether such as diethyl ether or tetrahydrofuran as inert solvents within a temperature range from 0° C. to +80° C.

Suitable inert solvents for process steps (VII) (I-2) and (IX) (I-4) are especially ethers such as diethyl ether, methyl tert-butyl ether, 1,2-dimethoxyethane, tetrahydrofuran and dioxane, or dipolar aprotic solvents such as dimethylformamide (DMF), dimethyl sulfoxide (DMSO), N-methylpyrrolidinone (NMP) and acetonitrile. It is equally possible to use mixtures of these solvents. Preference is given to using dimethylformamide. The reactions are effected generally within a temperature range from +20° C. to +150° C., preferably at +40° C. to +100° C.

The hydrolysis of the nitriles (I-2) to the carboxylic acids (VIII) is preferably performed with aqueous solutions of alkali metal or alkaline earth metal hydroxides such as lithium, sodium, potassium, calcium or barium hydroxide. Suitable cosolvents are alcohols such as methanol, ethanol, n-propanol, isopropanol, n-butanol or tert-butanol, ethers such as diethyl ether, tetrahydro-furan, dioxane or 1,2-dimethoxyethane, other solvents such as acetone, dimethylformamide (DMF) or dimethyl sulfoxide (DMSO), or mixtures of these solvents. The hydrolysis is effected generally within a temperature range from +50° C. to +150° C., preferably at +60° C. to +100° C.

The compounds of the formulae (II), (III) and (IV) are commercially available, known from the literature or can be prepared in analogy to literature processes.

The preparation of the inventive compounds can be illustrated by the following synthesis schemes:

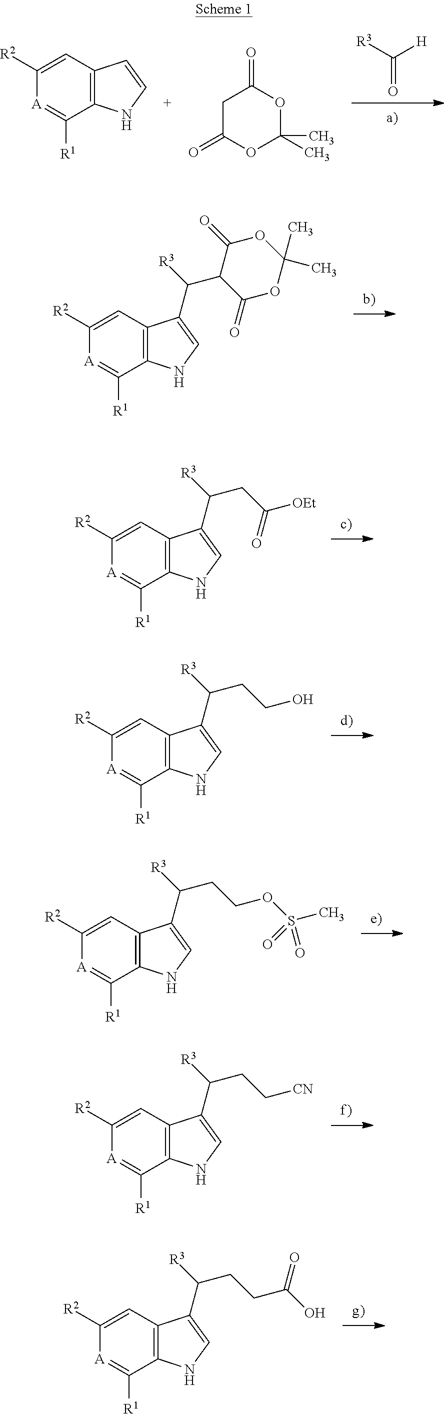

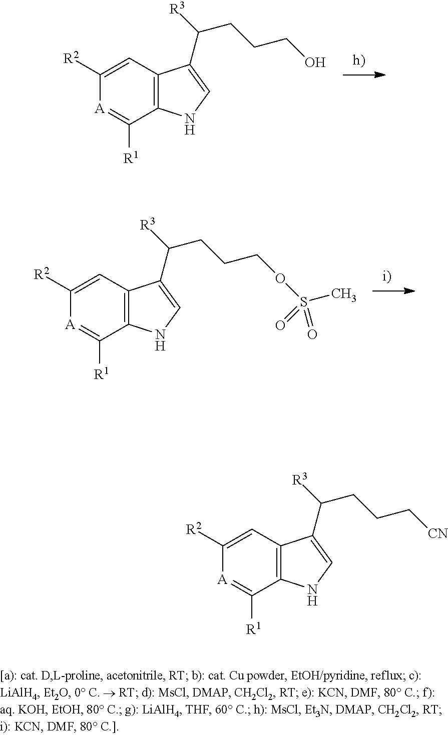

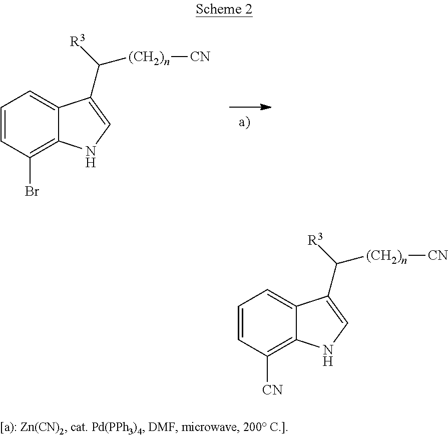

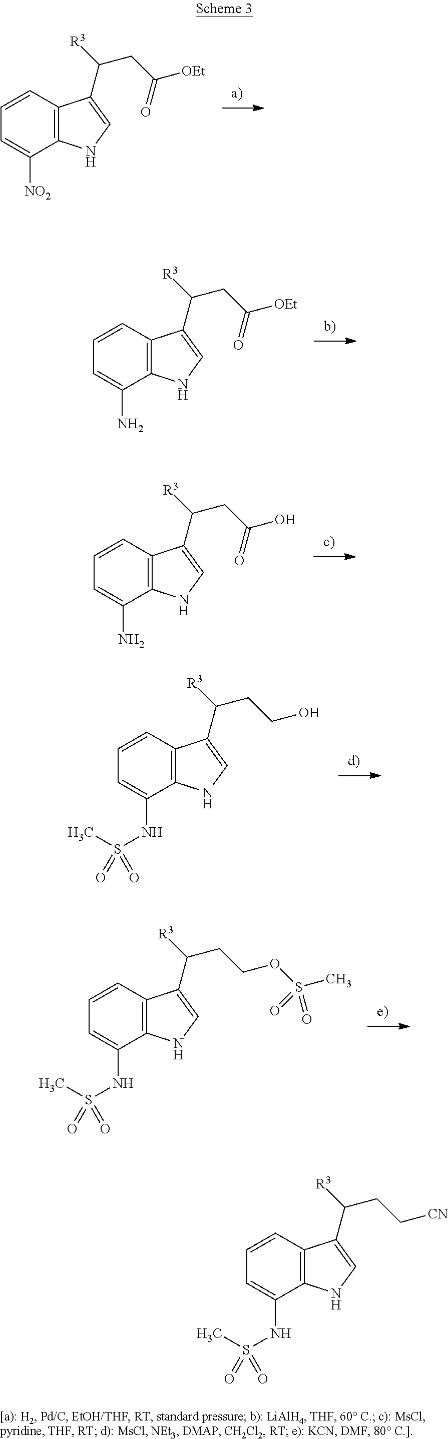

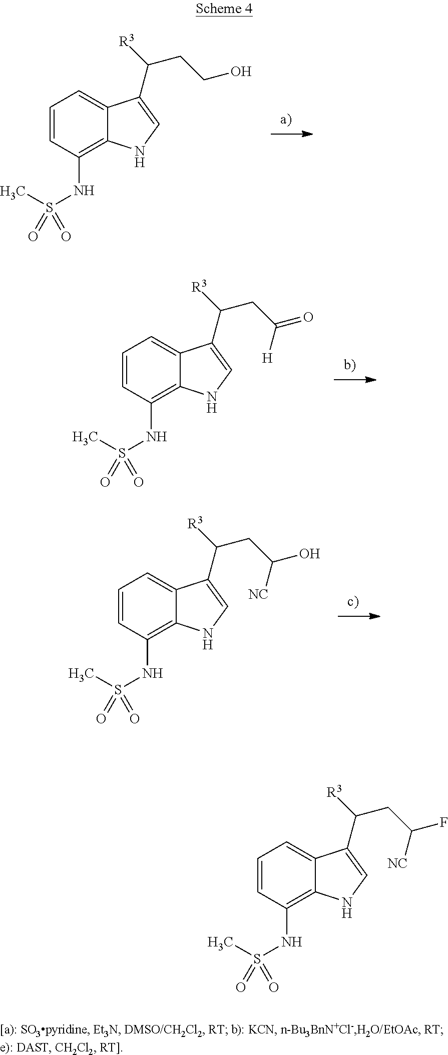

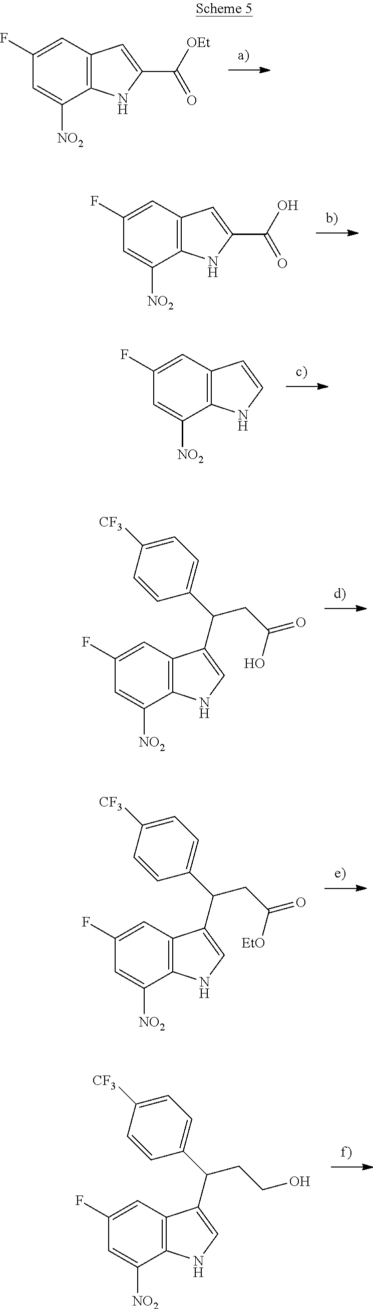

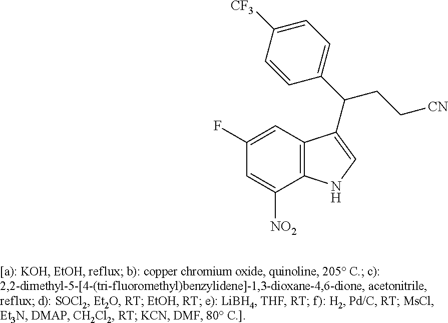

The inventive compounds are potent and selective antagonists of the mineralocorticoid receptor and exhibit an unforeseeable, valuable spectrum of pharmacological action. They are therefore suitable for use as medicaments for treatment and/or prophylaxis of diseases in man and animals.

The inventive compounds are suitable for the prophylaxis and/or treatment of various disorders and disease-related conditions, especially of disorders characterized either by an increase in the aldosterone concentration in the plasma or by a change in the aldosterone plasma concentration relative to the renin plasma concentration, or associated with these changes. Examples include: idiopathic primary hyperaldosteronism, hyperaldosteronism associated with adrenal hyperplasia, adrenal adenomas and/or adrenal carcinomas, hyperaldosteronism associated with cirrhosis of the liver, hyperaldosteronism associated with heart failure, and (relative) hyperaldosteronism associated with essential hypertension.

The inventive compounds are also suitable, because of their mechanism of action, for the prophylaxis of sudden cardiac death in patients at increased risk of dying of sudden cardiac death. These are especially patients suffering, for example, from one of the following disorders: primary and secondary hypertension, hypertensive heart disease with or without congestive heart failure, treatment-resistant hypertension, acute and chronic heart failure, coronary heart disease, stable and unstable angina pectoris, myocardial ischemia, myocardial infarction, dilative cardiomyopathies, inherited primary cardiomyopathies, for example Brugada syndrome, cardiomyopathies caused by Chagas disease, shock, arteriosclerosis, atrial and ventricular arrhythmia, transient and ischemic attacks, stroke, inflammatory cardiovascular disorders, peripheral and cardiac vascular disorders, peripheral blood flow disturbances, arterial occlusive disorders such as intermittent claudication, asymptomatic left-ventricular dysfunction, myocarditis, hypertrophic changes to the heart, pulmonary hypertension, spasms of the coronary arteries and peripheral arteries, thromboses, thromboembolic disorders, and vasculitis.

The inventive compounds can also be used for the prophylaxis and/or treatment of edema formation, for example pulmonary edema, renal edema or heart failure-related edema, and of restenoses such as following thrombolysis therapies, percutaneous transluminal angioplasties (PTA) and transluminal coronary angioplasties (PTCA), heart transplants and bypass operations.

The inventive compounds can additionally be used for the prophylaxis and/or treatment of erectile dysfunction.

The inventive compounds are further suitable for use as a potassium-saving diuretic and for electrolyte disturbances, for example hypercalcemia, hypernatremia or hypokalemia, including genetically related forms such as Gitelman or Banter syndrome.

The inventive compounds are equally suitable for treatment of renal disorders, such as acute and chronic renal failure, hypertensive renal disease, arteriosclerotic nephritis (chronic and interstitial), nephrosclerosis, chronic renal insufficiency and cystic renal disorders, for prevention of renal damage which can be caused, for example, by immunosuppressives such as cyclosporin A in the case of organ transplants, and for renal cancer.

The inventive compounds can additionally be used for the prophylaxis and/or treatment of diabetes mellitus and diabetic sequelae, for example neuropathy, nephropathy and cardiomyopathy.

The inventive compounds can further be used for the prophylaxis and/or treatment of eye disorders, especially forms based on angiogenesis and neovascularization, for example neonatal retinopathy, diabetic retinopathy, and age-related macular degeneration and glaucoma.

The inventive compounds can also be used for the prophylaxis and/or treatment of microalbuminuria, for example caused by diabetes mellitus or high blood pressure, and of proteinuria.

The inventive compounds are also suitable for the prophylaxis and/or treatment of disorders associated either with an increase in the plasma glucocorticoid concentration or with a local increase in the concentration of glucocorticoids in tissue (e.g. of the heart). Examples include: adrenal dysfunctions leading to overproduction of glucocorticoids (Cushing's syndrome), adrenocortical tumors with resulting overproduction of glucocorticoids, and pituitary tumors which autonomously produce ACTH (adrenocorticotropic hormone) and thus lead to adrenal hyperplasias with resulting Cushing's disease.

The inventive compounds can additionally be used for the prophylaxis and/or treatment of obesity, of metabolic syndrome and of obstructive sleep apnea.

The inventive compounds can also be used for the prophylaxis and/or treatment of inflammatory disorders caused for example by viruses, spirochetes, fungi, bacteria or mycobacteria, and of inflammatory disorders of unknown etiology, such as polyarthritis, lupus erythematosus, peri- or polyarteritis, dermatomyositis, scleroderma and sarcoidosis.

The inventive compounds can further be employed for the treatment of central nervous disorders such as depression, states of anxiety, and chronic pain, especially migraine, and for neurodegenerative disorders such as Alzheimer's disease and Parkinson's syndrome.

The inventive compounds are also suitable for the prophylaxis and/or treatment of vascular damage, for example following procedures such as percutaneous transluminal coronary angioplasty (PTCA), implantation of stents, coronary angioscopy, reocclusion or restenosis following bypass operations, and for endothelial dysfunction, for Raynaud's disease, for thromboangiitis obliterans (Buerger's syndrome) and for tinnitus syndrome.

The inventive compounds are also suitable for the prophylaxis and/or treatment of gynecological disorders such as endometriosis, leiomyomas of the uterus, dysfunctional bleeding and dysmenorrhea.

The present invention further provides for the use of the inventive compounds for treatment and/or prevention of disorders, especially the aforementioned disorders.

The present invention further provides for the use of the inventive compounds for production of a medicament for treatment and/or prevention of disorders, especially the aforementioned disorders.

The present invention further provides a method for treatment and/or prevention of disorders, especially the aforementioned disorders, using an effective amount of at least one of the inventive compounds.

The present invention further provides the inventive compounds for use in a method for treatment and/or prophylaxis of aldosteronism, high blood pressure, acute and chronic heart failure, the consequences of heart failure, liver cirrhosis, kidney failure and stroke.

The inventive compounds can be employed alone or, if required, in combination with other active ingredients. The present invention therefore further provides medicaments comprising at least one of the inventive compounds and one or more further active ingredients, especially for treatment and/or prevention of the aforementioned disorders. Preferred examples of suitable active ingredient combinations include:
active ingredients which lower blood pressure, for example and with preference from the group of calcium antagonists, angiotensin AII antagonists, ACE inhibitors, endothelin antagonists, renin inhibitors, alpha-receptor blockers, beta-receptor blockers and Rho kinase inhibitors;
diuretics, especially loop diuretics, and thiazides and thiazide-like diuretics;
antithrombotic agents, for example and with preference from the group of platelet aggregation inhibitors, of anticoagulants or of profibrinolytic substances;
active ingredients which alter lipid metabolism, for example and with preference from the group of thyroid receptor agonists, cholesterol synthesis inhibitors, preferred examples being HMG-CoA reductase inhibitors or squalene synthesis inhibitors, of ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein(a) antagonists;
organic nitrates and NO donors, for example sodium nitroprusside, nitroglycerin, isosorbide mononitrate, isosorbide dinitrate, molsidomine or SIN-1, and inhaled NO;
compounds having a positive inotropic effect, for example cardiac glycosides (digoxin), beta-adrenergic and dopaminergic agonists such as isoproterenol, adrenaline, noradrenaline, dopamine and dobutamine;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil and tadalafil, and PDE 3 inhibitors such as aminone and milrinone;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
calcium sensitizers, a preferred example being levosimendan;
NO- and heme-independent activators of guanylate cyclase, such as especially cinaciguat and the compounds described in WO 01/19355, WO 01/19776, WO 01/19778, WO 01/19780, WO 02/070462 and WO 02/070510;
NO-independent but heme-dependent stimulators of guanylate cyclase, such as especially riociguat and the compounds described in WO 00/06568, WO 00/06569, WO 02/42301 and WO 03/095451;
modulators of adenosine receptors, especially adenosine A1 antagonists such as KW-3902, SLV-320 or BG-9928 (Adentri);
vasopressin receptor antagonists, for example conivaptan (Vaprisol), tolvaptan, satavaptan, lixivaptan, relcovaptan, RWJ-339489 or RWJ-351647;
inhibitors of human neutrophil elastase (HNE), for example sivelestat or DX-890 (Reltran);
compounds which inhibit the signal transduction cascade, for example tyrosine kinase inhibitors, especially sorafenib, imatinib, gefitinib and erlotinib; and/or
compounds which influence the energy metabolism of the heart, preferred examples being etomoxir, dichloroacetate, ranolazine or trimetazidine.

In a preferred embodiment of the invention, the compounds of the invention are administered in combination with a diuretic, preferred examples being furosemide, bumetanide, torsemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorophenamide, methazolamide, glycerol, isosorbide, mannitol, amiloride or triamterene.

Agents which lower blood pressure are preferably understood to mean compounds from the group of calcium antagonists, angiotensin AII antagonists, ACE inhibitors, endothelin antagonists, renin inhibitors, alpha-receptor blockers, beta-receptor blockers, Rho kinase inhibitors, and the diuretics.

In a preferred embodiment of the invention, the inventive compounds are administered in combination with a calcium antagonist, preferred examples being nifedipine, amlodipine, verapamil or diltiazem.

In a preferred embodiment of the invention, the inventive compounds are administered in combination with an angiotensin AII antagonist, preferred examples being losartan, candesartan, valsartan, telmisartan or embusartan.

In a preferred embodiment of the invention, the inventive compounds are administered in combination with an ACE inhibitor, preferred examples being enalapril, captopril, lisinopril, ramipril, delapril, fosinopril, quinopril, perindopril or trandopril.

In a preferred embodiment of the invention, the inventive compounds are administered in combination with an endothelin antagonist, preferred examples being bosentan, darusentan, ambrisentan or sitaxsentan.

In a preferred embodiment of the invention, the inventive compounds are administered in combination with a renin inhibitor, preferred examples being aliskiren, SPP-600, SPP-635, SPP-676, SPP-800 or SPP-1148.

In a preferred embodiment of the invention, the inventive compounds are administered in combination with an alpha-1 receptor blocker, a preferred example being prazosin.

In a preferred embodiment of the invention, the inventive compounds are administered in combination with a beta receptor blocker, preferred examples being propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the inventive compounds are administered in combination with a Rho kinase inhibitor, preferred examples being fasudil, Y-27632, SLx-2119, BF-66851, BF-66852, BF-66853, KI-23095 or BA-1049.

Antithrombotic agents (antithrombotics) are preferably understood to mean compounds from the group of platelet aggregation inhibitors, of anticoagulants or of profibrinolytic substances.

In a preferred embodiment of the invention, the inventive compounds are administered in combination with a platelet aggregation inhibitor, preferred examples being aspirin, clopidogrel, ticlopidin or dipyridamol.

In a preferred embodiment of the invention, the inventive compounds are administered in combination with a thrombin inhibitor, preferred examples being ximelagatran, melagatran, bivalirudin or clexane.

In a preferred embodiment of the invention, the inventive compounds are administered in combination with a GPIIb/IIIa antagonist, preferred examples being tirofiban or abciximab.

In a preferred embodiment of the invention, the inventive compounds are administered in combination with a factor Xa inhibitor, preferred examples being rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the inventive compounds are administered in combination with heparin or a low molecular weight (LMW) heparin derivative.

In a preferred embodiment of the invention, the inventive compounds are administered in combination with a vitamin K antagonist, a preferred example being coumarin.

Active ingredients which alter lipid metabolism are preferably understood to mean compounds from the group of CETP inhibitors, thyroid receptor agonists, cholesterol synthesis inhibitors such as HMG-CoA reductase inhibitors or squalene synthesis inhibitors, of ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and lipoprotein(a) antagonists.

In a preferred embodiment of the invention, the inventive compounds are administered in combination with a CETP inhibitor, preferred examples being dalcetrapib, BAY 60-5521, anacetrapib or CETP vaccine (CETi-1).

In a preferred embodiment of the invention, the inventive compounds are administered in combination with a thyroid receptor agonist, preferred examples being D-thyroxin, 3,5,3′-triiodothyronin (T3), CGS 23425 or axitirome (CGS 26214).

In a preferred embodiment of the invention, the inventive compounds are administered in combination with a HMG-CoA reductase inhibitor from the class of the statins, preferred examples being lovastatin, simvastatin, pravastatin, fluvastatin, atorvastatin, rosuvastatin or pitavastatin.

In a preferred embodiment of the invention, the inventive compounds are administered in combination with a squalene synthesis inhibitor, preferred examples being BMS-188494 or TAK-475.

In a preferred embodiment of the invention, the inventive compounds are administered in combination with an ACAT inhibitor, preferred examples being avasimibe, melinamide, pactimibe, eflucimibe or SMP-797.

In a preferred embodiment of the invention, the inventive compounds are administered in combination with an MTP inhibitor, preferred examples being implitapide, BMS-201038, R-103757 or JTT-130.

In a preferred embodiment of the invention, the inventive compounds are administered in combination with a PPAR-gamma antagonist, preferred examples being pioglitazone or rosiglitazone.

In a preferred embodiment of the invention, the inventive compounds are administered in combination with a PPAR-delta antagonist, preferred examples being GW-501516 or BAY 68-5042.

In a preferred embodiment of the invention, the inventive compounds are administered in combination with a cholesterol absorption inhibitor, preferred examples being ezetimibe, tiqueside or pamaqueside.

In a preferred embodiment of the invention, the inventive compounds are administered in combination with a lipase inhibitor, a preferred example being orlistat.

In a preferred embodiment of the invention, the inventive compounds are administered in combination with a polymeric bile acid adsorbent, preferred examples being cholestyramine, colestipol, colesolvam, CholestaGel or colestimide.

In a preferred embodiment of the invention, the inventive compounds are administered in combination with a bile acid reabsorption inhibitor, preferred examples being ASBT (=IBAT) inhibitors, for example AZD-7806, S-8921, AK-105, BARI-1741, SC-435 or SC-635.

In a preferred embodiment of the invention, the inventive compounds are administered in combination with a lipoprotein(a) antagonist, preferred examples being gemcabene calcium (CI-1027) or nicotinic acid.

The present invention further provides medicaments which comprise at least one inventive compound, typically together with one or more inert, nontoxic, pharmaceutically suitable excipients, and the use thereof for the aforementioned purposes.

The inventive compounds may act systemically and/or locally. For this purpose, they can be administered in a suitable manner, for example by the oral, parenteral, pulmonary, nasal, sublingual, lingual, buccal, rectal, dermal, transdermal, conjunctival or otic route, or as implant or stent.

The inventive compounds can be administered in administration forms suitable for these administration routes.

Suitable administration forms for oral administration are those which work according to the prior art, which release the inventive compounds rapidly and/or in a modified manner and which contain the inventive compounds in crystalline and/or amorphized and/or dissolved form, for example tablets (uncoated or coated tablets, for example with gastric juice-resistant or retarded-dissolution or insoluble coatings which control the release of the inventive compound), tablets or films/oblates which disintegrate rapidly in the oral cavity, films/lyophilizates or capsules (for example hard or soft gelatin capsules), sugar-coated tablets, granules, pellets, powders, emulsions, suspensions, aerosols or solutions.

Parenteral administration can be accomplished with avoidance of an absorption step (e.g. by an intravenous, intraarterial, intracardiac, intraspinal or intralumbar route) or with inclusion of an absorption (e.g. by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Oral and parenteral administration are preferred, in particular oral and intravenous administration.

The inventive compounds can be converted to the administration forms mentioned. This can be done in a manner known per se by mixing with inert, nontoxic, pharmaceutically suitable excipients. These auxiliary substances include carrier substances (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavor and/or odor correctants.

In general, it has been found to be advantageous in the case of parenteral administration to administer amounts of from about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight.

It may nevertheless be necessary in some cases to deviate from the stated amounts, specifically as a function of the body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of relatively large amounts, it may be advisable to divide these into several individual doses over the course of the day.

The working examples which follow illustrate the invention. The invention is not limited to the examples.

The percentages in the following tests and examples are, unless stated otherwise, percentages by weight; parts are parts by weight. Solvent ratios, dilution ratios and concentration data for liquid/liquid solutions are in each case based on volume.

### A. EXAMPLES

### Abbreviations and Acronyms

Ac acetyl
Bn benzyl
Bu butyl
cat. catalytic
CI chemical ionization (in MS)
DAST diethylaminosulfur trifluoride

### DMAP 4-N,N-dimethylaminopyridine

DMF dimethylformamide
DMSO dimethyl sulfoxide
EI electron impact ionization (in MS)
eq. equivalent(s)
ESI electrospray ionization (in MS)
Et ethyl
EtOAc ethyl acetate
sat. saturated
h hour(s)
HPLC high-pressure, high-performance liquid chromatography
conc. concentrated
LC-MS liquid chromatography-coupled mass spectrometry
Me methyl
min minute(s)
Ms methanesulfonyl (mesyl)
MS mass spectrometry
NMR nuclear magnetic resonance spectrometry
Pd/C palladium on activated carbon
Ph phenyl
RT room temperature
Rₜ retention time (in HPLC)
THF tetrahydrofuran
UV ultraviolet spectrometry
v/v volume to volume ratio (of a solution)
aq. aqueous, aqueous solution

### LC-MS and HPLC Methods:

Method 1 (LC-MS): MS instrument type: Micromass ZQ; HPLC instrument type: Waters Alliance 2795; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 2 (LC-MS): MS instrument type: Waters ZQ; HPLC instrument type: Waters Alliance 2795; column: Merck Chromolith RP18e, 100 mm×3 mm; eluent A: 1 l of water+0.5 ml of 50% formic acid, eluent B: 1 l of acetonitrile+0.5 ml of 50% formic acid; gradient. 0.0 min 90% A→2 min 65% A→4.5 min 5% A→6 min 5% A; flow rate: 2 ml/min; oven: 40° C.; UV detection: 210 nm.

Method 3 (LC-MS): Instrument: Micromass Quattro LCZ with HPLC Agilent series 1100; column: Phenomenex Onyx Monolithic C18, 100 mm×3 mm; eluent A: 1 l of water+0.5 ml of 50% formic acid, eluent B: 1 l of acetonitrile+0.5 ml of 50% formic acid; gradient: 0.0 min 90% A→2 min 65% A→4.5 min 5% A→6 min 5% A; flow rate: 2 ml/min; oven: 40° C.; UV detection: 208-400 nm.

Method 4 (HPLC): Instrument: HP 1100 with DAD detection; column: Kromasil 100 RP-18, 60 mm×2.1 mm, 3.5 μm; eluent A: 5 ml of HClO₄ (70%)/1 of water, eluent B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

Method 5 (HPLC): Instrument: HP 1100 with DAD detection; column: Kromasil 100 RP-18, 60 mm×2.1 mm, 3.5 μm; eluent A: 5 ml of HClO₄ (70%)/1 of water, eluent B: acetonitrile; gradient: 0 min 2% B→0.5 min 2% B→4.5 min 90% B→6.5 min 90% B→6.7 min 2% B 7.5 min 2% B; flow rate: 0.75 ml/min; column temperature: 30° C.; UV detection: 210 nm.

Method 6 (LC-MS): Instrument: Micromass Quattro LCZ with HPLC Agilent series 1100; column: Phenomenex Gemini 3μ, 30 mm×3.00 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min 2.5 min/3.0 min/4.5 min 2 ml/min; oven: 50° C.; UV detection: 208-400 nm.

Method 7 (LC-MS): MS instrument type: Micromass ZQ; HPLC instrument type: Waters Alliance 2795; column: Phenomenex Synergi 2μ MAX-RP 100A Mercury 20 mm×4 mm; eluent A: 1 l of water+0.5 ml of 50% formic acid, eluent B: 1 l of acetonitrile+0.5 ml of 50% formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 8 (LC-MS): Instrument: Micromass QuattroPremier with Waters HPLC Acquity; column: Thermo Hypersil GOLD 1.9μ, 50 mm×1 mm; eluent A: 1 l of water+0.5 ml of 50% formic acid, eluent B: 1 l of acetonitrile+0.5 ml of 50% formic acid; gradient: 0.0 min 90% A→0 l min 90% A→1.5 min 10% A→2.2 min 10% A; flow rate: 0.33 ml/min; oven: 50° C.; UV detection: 210 nm.

Method 9 (LC-MS): MS instrument type: Micromass ZQ; HPLC instrument type: HP 1100 Series; UV DAD; column: Phenomenex Gemini 3μ, 30 mm×3.00 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min 2.5 min/3.0 min/4.5 min 2 ml/min; oven: 50° C.; UV detection: 210 nm.

### Starting Compounds and Intermediates:

### Example 1A

### 5-{(7-Ethyl-1H-indol-3-yl)[4-(trifluoromethyl)phenyl]methyl}-2,2-dimethyl-1,3-dioxane-4,6-dione

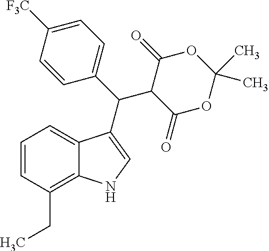

To a solution of 40.0 g of 7-ethylindole (275 mmol), 39.7 g of Meldrum's acid (275 mmol) and 48.0 g of 4-trifluoromethylbenzaldehyde (275 mmol) in 400 ml of acetonitrile were added 1.6 g of D,L-proline (14 mmol). The mixture was stirred at RT overnight. The precipitated solid was then filtered off with suction, washed with acetonitrile and dried under high vacuum. This gave 115 g (94% of theory) of the target compound.

¹H-NMR (400 MHz, DMSO-d₆): δ=1.27 (t, 3H), 1.61 (s, 3H), 1.85 (s, 3H), 2.85 (q, 2H), 5.36 (d, 1H), 5.46 (br. s, 1H), 6.82-6.88 (m, 2H), 6.91 (d, 1H), 7.05 (d, 1H), 7.14 (m_{c}, 1H), 7.52 (d, 2H), 7.61 (d, 2H), 11.04 (s, 1H).

LC-MS (method 1): Rₜ=2.74 min; MS (ESIneg): m/z=444.3 [M−H]⁻.

### Example 2A

### Ethyl 3-(7-ethyl-1H-indol-3-yl)-3-[4-(trifluoromethyl)phenyl]propionate

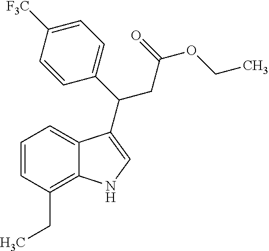

To 25.0 g of the compound from example 1A (56.1 mmol) in 100 ml of pyridine and 20 ml of ethanol was added 0.18 g of copper powder (˜150 mesh, 2.1 mmol). The mixture was heated under reflux overnight. The solvent was then removed under reduced pressure and the residue was purified by chromatography using a silica gel column (eluent: cyclohexane/ethyl acetate 100:1→3:1). 20.5 g (94% of theory) of the target compound were obtained.

¹H-NMR (200 MHz, DMSO-d₆): δ=1.07 (t, 3H), 1.25 (t, 3H), 2.85 (q, 2H), 3.14+3.24 (AB signal, split in addition to the d, 2H), 4.00 (q, 2H), 4.75 (t, 1H), 6.80-6.91 (m, 2H), 7.23 (dd, 1H), 7.38 (d, 1H), 7.61 (s, 4H), 11.0 (s, 1H).

### Example 3A

### 3-(7-Ethyl-1H-indol-3-yl)-3-[4-(trifluoromethyl)phenyl]propyl methanesulfonate

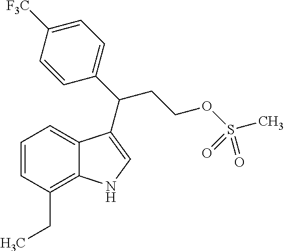

To 500 mg of the compound from example 1 in 5 ml of dichloromethane were added 0.341 ml of triethylamine (247 mg, 2.45 mmol) and 18 mg of 4-N,N-dimethylaminopyridine (0.14 mmol). The mixture was left to stir at RT for 5 min and then 0.223 ml of methanesulfonyl chloride (329 mg, 2.88 mmol) was added. The reaction mixture was stirred at RT overnight and then admixed with 15 ml of ethyl acetate. After extraction with 20 ml each of 1 N hydrochloric acid, water and sat. aq. sodium chloride solution, the organic phase was dried over magnesium sulfate and freed of the solvent under reduced pressure. The residue was used without further purification.

LC-MS (method 2): Rₜ=3.95 min; MS (ESIpos): m/z=426.2 [M+H]⁺.

### Example 4A

### 4-(7-Ethyl-1H-indol-3-yl)-4-(4-trifluoromethylphenyl)butyric acid

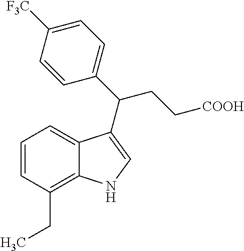

958 mg of the compound from example 2 (2.69 mmol) were initially charged in 9 ml of ethanol. 603 mg of potassium hydroxide in 4.5 ml of water were added and the mixture was stirred at 80° C. for 6 h. The mixture was then cooled, poured onto ice-water and adjusted to pH 3 with 1 N hydrochloric acid. It was extracted twice with 20 ml of ethyl acetate. The combined organic phases were extracted with 20 ml of sat. aq. sodium chloride solution, dried over magnesium sulfate and freed of the solvent under reduced pressure. The residue was purified using a silica gel frit (eluent: cyclohexane/ethyl acetate 1:1). 927 mg (90% of theory) of the target compound were obtained.

¹H-NMR (400 MHz, DMSO-d₆): δ=1.23 (t, 3H), 2.10-2.30 (m, 3H), 2.35-2.46 (m, 1H), 4.03 (q, 2H), 4.26 (t, 1H), 6.79-6.88 (m, 2H), 7.19 (d, 1H), 7.32 (d, 1H), 7.54 (d, 2H), 7.62 (d, 2H), 10.93 (s, 1H), 12.07 (s, 1H).

MS (CIpos): m/z=393.0 [M+NH₄]⁺.

### Example 5A

### Ethyl 3-(7-nitro-1H-indol-3-yl)-3-[4-(trifluoromethyl)phenyl]propionate

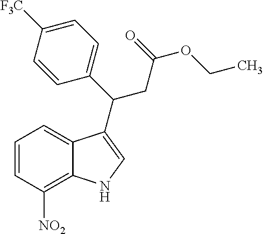

The title compound was prepared proceeding from 7-nitroindole analogously to the synthesis of the compound from example 2A.

¹H-NMR (400 MHz, DMSO-d₆): δ=1.03 (t, 3H), 3.18+3.25 (AB signal, split in addition to the d, 2H), 3.96 (q, 2H), 4.85 (t, 1H), 7.16 (dd, 1H), 7.60-7.66 (m, 5H), 7.97 (d, 1H), 8.06 (d, 1H), 11.85 (s, 1H).

HPLC (method 4): Rₜ=5.20 min; MS (ESIneg): m/z=405.2 [M−H]⁻.

### Example 6A

### Ethyl 3-(7-amino-1H-indol-3-yl)-3-[4-(trifluoromethyl)phenyl]propionate

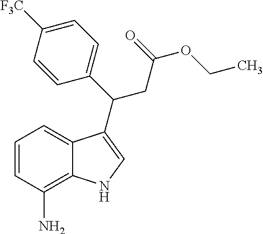

3.66 g of the compound from example 5A (9.01 mmol) in 30 ml of ethanol and 60 ml of THF were admixed with 400 mg of palladium on carbon and hydrogenated under standard pressure at RT overnight. The mixture was filtered through Celite and washed with ethanol, and the filtrate was concentrated under reduced pressure. The residue was purified by chromatography using a silica gel column (eluent: dichloromethane→dichloromethane/methanol 100:1). 3.32 g (98% of theory) of the target compound were obtained.

¹H-NMR (400 MHz, DMSO-d₆): δ=1.05 (t, 3H), 3.08+3.16 (AB signal, split in addition to the d, 2H), 3.96 (q, 2H), 4.65 (t, 1H), 4.96 (s, 2H), 6.26 (dd, 1H), 6.58-6.65 (m, 2H), 7.26 (d, 1H), 7.54 (d, 2H), 7.59 (d, 2H), 10.51 (s, 1H).

HPLC (method 5): Rₜ=4.28 min.

### Example 7A

### 3-[4-(Trifluoromethyl)phenyl]-3-{7-[(methylsulfonyl)amino]-1H-indol-3-yl}propyl methanesulfonate

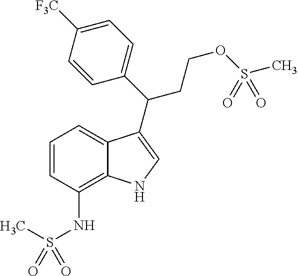

To 45.0 mg of the compound from example 16 (0.109 mmol) in 0.5 ml of dichloromethane were added 1.3 mg of 4-N,N-dimethylaminopyridine (0.011 mmol) and 26 μl of triethylamine (19 mg, 0.19 mmol). The mixture was left to stir for 5 min and then 13 μl of methanesulfonyl chloride (19 mg, 0.16 mmol) were added. After stirring at RT overnight, 5 ml of ethyl acetate and 5 ml of water were added. The organic phase was extracted with 5 ml each of 1 N hydrochloric acid, water and sat. aq. sodium chloride solution. The organic phase was dried over magnesium sulfate and freed of the solvent under reduced pressure. The residue was purified by chromatography using a silica gel column (eluent: dichloromethane/methanol 100:1). 44.9 mg (76% of theory) of the target compound were obtained.

LC-MS (method 3): Rₜ=3.58 min; MS (ESIpos): m/z=491.2 [M+H]⁺.

### Example 8A

### N-(3-{3-Oxo-1-[4-(trifluoromethyl)phenyl]propyl}-1H-indol-7-yl)methanesulfonamide

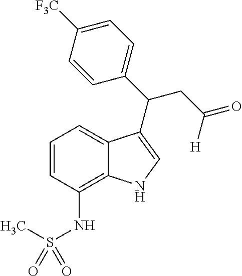

2.80 g of sulfur trioxide-pyridine complex (17.6 mmol) were dissolved at 0° C. in 12 ml of DMSO/dichloromethane (1:1). After stirring for 15 min, 1.45 g of the compound from example 16 (3.52 mmol) and 5.9 ml of triethylamine (4.27 g, 42.2 mmol) were added, and the solution was warmed up to RT within 30 min and stirred at RT for 1 h. Subsequently, 20 ml each of dichloromethane and water were added and the phases were separated. The organic phase was washed twice with water, dried over sodium sulfate and freed of the solvent under reduced pressure. The residue was purified by means of preparative HPLC (eluent: acetonitrile/water, gradient 30:70→98:2). This gave 0.67 g (87% purity, 40% of theory) of the target compound.

LC-MS (method 7): Rₜ=1.96 min; MS (ESIneg): m/z=409.3 [M−H]⁻.

### Example 9A

### N-1H-Indol-7-ylmethanesulfonamide

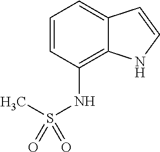

4.34 g (32.8 mmol) of 7-amino-1H-indole were initially charged in 120 ml of dichloromethane, 3.76 g (32.8 mmol) of methanesulfonyl chloride and 2.60 g (32.8 mmol) of pyridine were added, and the mixture was stirred at RT for three days. After concentrating to one third of the volume, ethyl acetate was added and the mixture was washed successively with 1 M hydrochloric acid, water and saturated sodium chloride solution. The organic phase was dried over magnesium sulfate, filtered and concentrated. The residue was purified by means of flash chromatography (eluent: toluene/ethyl acetate 9:1) to obtain 5.63 g (82% of theory) of the title compound.

¹H-NMR (400 MHz, DMSO-d₆): δ=2.97 (s, 3H), 6.46 (t, 1H), 6.98 (t, 1H), 7.06 (d, 1H), 7.36 (t, 1H), 7.41 (d, 1H), 9.31 (s, 1H), 10.8 (s, 1H).

LC-MS (method 8): Rₜ=0.81 min; MS (ESIpos): m/z=211 [M+H]⁺.

### Example 10A

### N-(3-{(2,2-Dimethyl-4,6-dioxo-1,3-dioxan-5-yl)[2-fluoro-4-(trifluoromethyl)phenyl]methyl}-1H-indol-7-yl)methanesulfonamide

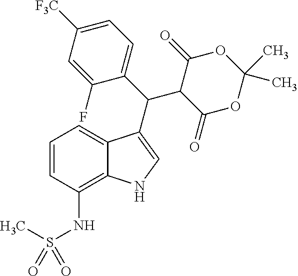

The title compound was prepared proceeding from 1.00 g (4.76 mmol) of the compound from example 9A analogously to the synthesis of the compound from example 1A. The crude product was purified first by means of flash chromatography on silica gel (eluent: toluene/ethyl acetate gradient) and then by means of preparative HPLC (RP18 column; eluent: acetonitrile/water gradient with addition of 0.1% formic acid). This gave 1.59 g (63% of theory) of the title compound.

LC-MS (method 8): Rₜ=1.23 min; MS (ESIneg): m/z=527 [M−H]⁻.

The compounds listed in the table which follows were prepared analogously to the synthesis of the compound from example 10A. In a departure from the reaction for example 16A, the mixture was stirred first at 60° C. for 8 h and then at RT for two days:

| | | | Yield |
|---|---|---|---|
| Example | | Starting | (% of theory); analytical |
| No. | Structure | compound | data |
| 11A | 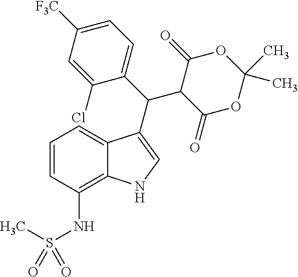 | 9A | 43% LC-MS (method 8): Rₜ = 1.26 min; MS (ESIneg): m/z = 543 [M − H]⁻. |
| 12A | 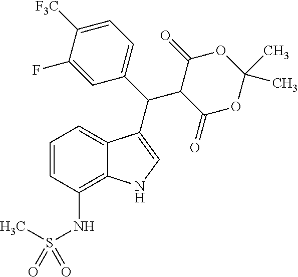 | 9A | 70% LC-MS (method 8): Rₜ = 1.24 min; MS (ESIneg): m/z = 527 [M − H]⁻ ¹H NMR (400 MHz, DMSO- d₆): δ = 1.64 (s, 3H), 1.87 (s, 3H), 3.00 (s, 3H), 5.42-5.47 (m, 2H), 6.94 (t, 1H), 7.07 (d, 1H), 7.16 (d, 1H), 7.21 (d, 1H), 7.31 (d, 1H), 7.46 (d, 1H), 7.66 (t, 1H), 9.40 (s, 1H), 10.9 (s, 1H). |
| 13A | 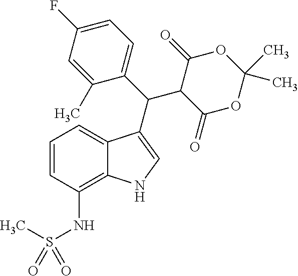 | 9A | 46% LC-MS (method 9): Rₜ = 2.59 min; MS (ESIpos): m/z = 475 [M + H]⁺. |
| 14A | 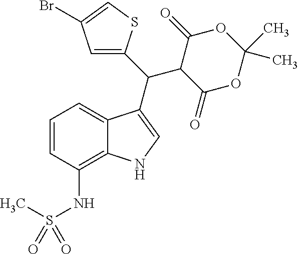 | 9A | 64% LC-MS (method 7): Rₜ = 1.97 min; MS (ESIneg): m/z =525 [M − H]⁻. |
| 15A | 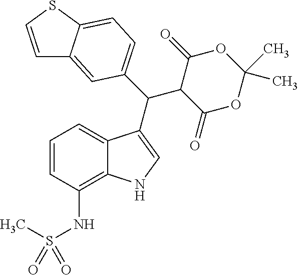 | 9A | 43% LC-MS (method 8): Rₜ = 1.23 min; MS (ESIneg): m/z = 497 [M − H]⁻. |
| 16A | 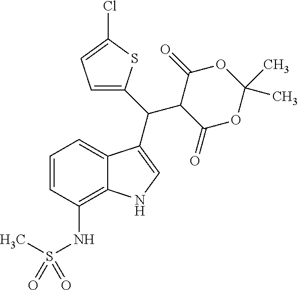 | 9A | 49% LC-MS (method 7): Rₜ = 1.99 min; MS (ESIneg): m/z =481 [M − H]⁻. |

### Example 17A

### Ethyl 3-[2-fluoro-4-(trifluoromethyl)phenyl]-3-{7-[(methylsulfonyl)amino]-1H-indol-3-yl}-propanoate

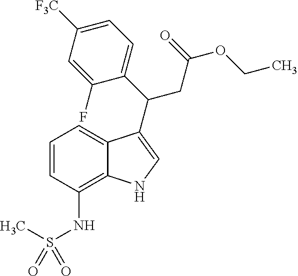

To 1.59 g (3.01 mmol) of the compound from example 10A in 21 ml of pyridine and 5.4 ml of ethanol were added 2 mg (0.03 mmol) of copper powder. The mixture was heated under reflux for 1 h. The solvent was then removed under reduced pressure, the residue was taken up in ethyl acetate and washed with 1 M hydrochloric acid, and the org. phase was dried over magnesium sulfate, filtered and concentrated. The residue was purified by chromatography using a silica gel column (eluent: cyclohexane/ethyl acetate gradient). 1.00 g (70% of theory) of the target compound was obtained.

¹H-NMR (400 MHz, DMSO-d₆): δ=1.04 (t, 3H), 2.96 (s, 3H), 3.22 (d, 2H), 3.97 (q, 2H), 4.98 (t, 1H), 6.94 (t, 1H), 7.04 (d, 1H), 7.27 (d, 1H), 7.38 (d, 1H), 7.49 (d, 1H), 7.59-7.68 (m, 2H), 9.30 (s, 1H), 10.8 (s, 1H).

LC-MS (method 8): Rₜ=1.33 min; MS (ESIpos): m/z=473 [M+H]⁺.

The compounds listed in the table which follows were prepared analogously to the synthesis of the compound from example 17A. In a departure therefrom, it was also possible to heat under reflux for 2 h. It was also possible as an alternative, and without further workup, to purify the reaction mixture directly by means of flash chromatography (eluent: cyclohexane/ethyl acetate gradient) and subsequent preparative HPLC (RP18 column; eluent: acetonitrile/water gradient).

| | | | Yield |
|---|---|---|---|
| Example | | Starting | (% of theory); analytical |
| No. | Structure | compound | data |
| 18A | 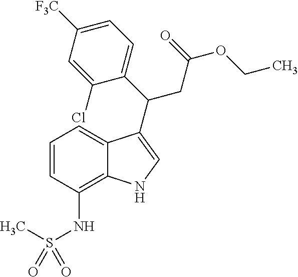 | 11A | 64% LC-MS (method 8): Rₜ = 1.39 min; MS (ESIpos): m/z = 489 [M + H]⁺ ¹H NMR (400 MHz, DMSO- d₆): δ = 1.05 (t, 3H), 2.97 (s, 3H), 3.13 (dd, 1H), 3.21 (dd, 1H), 3.97 (q, 2H), 5.17 (t, 1H), 6.94 (t, 1H), 7.04 (d, 1H), 7.25 (d, 1H), 7.35 (d, 1H), 7.60-7.66 (m, 2H), 7.85 (s, 1H), 9.31 (s, 1H), 10.8 (s, 1H). |
| 19A | 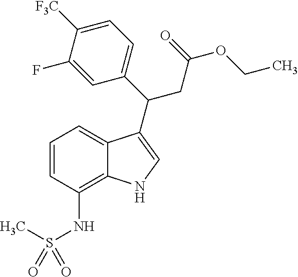 | 12A | 88% LC-MS (method 8): Rₜ = 1.33 min; MS (ESIpos): m/z = 473 [M + H]⁺ ¹H NMR (400 MHz, DMSO- d₆): δ = 1.04 (t, 3H), 2.96 (s, 3H), 3.12-3.26 (m, 2H), 3.97 (q, 2H), 4.76 (t, 1H), 6.93 (t, 1H), 7.03 (d, 1H), 7.35 (d, 1H), 7.41-7.46 (m, 2H), 7.57 (d, 1H), 7.65 (t, 1H), 9.29 (s, 1H), 10.8 (s, 1H). |
| 20A | 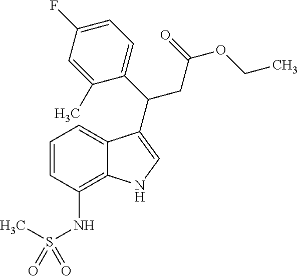 | 13A | 25% LC-MS (method 9): Rₜ = 2.45 min; MS (ESIpos): m/z = 419 [M + H]⁺ ¹H NMR (400 MHz, DMSO- d₆): δ = 1.03 (t, 3H), 2.43 (s, 3H), 2.96 (s, 3H), 2.98 (dd, 1H), 3.09 (dd, 1H), 3.95 (q, 2H), 4.81 (t, 1H), 6.87-6.94 (m, 2H), 6.98-7.04 (m, 2H), 7.14 (d, 1H), 7.19 (d, 1H), 7.24 (dd, 1H), 9.28 (s, 1H), 10.7 (s, 1H). |
| 21A | 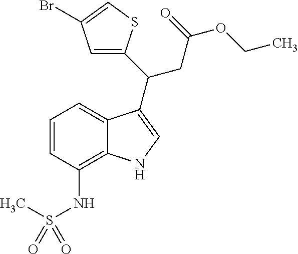 | 14A | 33% HPLC (method 4): R^{t} = 4.66 min; MS (ESIpos): m/z = 471 [M + H]⁺ ¹H NMR (400 MHz, DMSO- d₆): δ = 1.07 (t, 3H), 2.97 (s, 3H), 3.16 (d, 2H), 3.95-4.06 (m, 2H), 4.90 (t, 1H), 6.95 (t, 1H), 7.02-7.07 (m, 2H), 7.33 (d, 1H), 7.36 (d, 1H), 7.40 (d, 1H), 9.31 (s, 1H), 10.8 (d, 1H). |
| 22A | 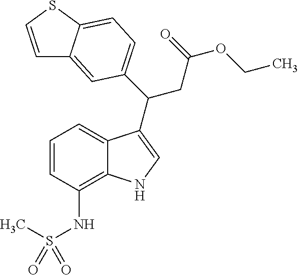 | 15A | 50% HPLC (method 5): Rₜ = 4.62 min; MS (ESIpos): m/z = 443 [M + H]⁺ ¹H NMR (400 MHz, DMSO- d₆): δ = 1.03 (t, 3H), 2.94 (s, 3H), 3.11 (dd, 1H), 3.22 (dd, 1H), 3.89-4.01 (m, 2H), 4.75 (t, 1H), 6.87 (t, 1H), 7.00 (d, 1H), 7.25 (d, 1H), 7.33-7.40 (m, 3H), 7.70 (d, 1H), 7.82- 7.87 (m, 2H), 9.27 (s, 1H), 10.7 (d, 1H). |
| 23A | 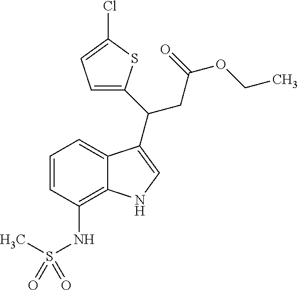 | 16A | 20% LC-MS (method 8): Rₜ = 1.30 min; MS (ESIneg): m/z = 425 [M − H]⁻ ¹H NMR (400 MHz, DMSO- d₆): δ = 1.08 (t, 3H), 2.98 (s, 3H), 3.13 (d, 2H), 4.00 (q, 2H), 4.85 (t, 1H), 6.87-6.91 (m, 2H), 6.95 (t, 1H), 7.05 (d, 1H), 7.32 (d, 1H), 7.35 (d, 1H), 9.31 (s, 1H), 10.8 (d, 1H). |

### Example 24A

### 5-Fluoro-7-nitro-1H-indole-2-carboxylic acid

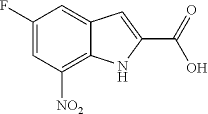

1.85 g (6.15 mmol) of ethyl 5-fluoro-7-nitro-1H-indole-2-carboxylate were initially charged in 20 ml of ethanol, admixed with 517 mg (9.22 mmol) of potassium hydroxide and stirred under reflux overnight. Subsequently, ethyl acetate was added, the mixture was extracted with 1 M sodium hydroxide solution and the pH of the aqueous phase was adjusted to pH 2 with hydrochloric acid.

The mixture was extracted repeatedly with ethyl acetate, the organic phase was washed with saturated sodium chloride solution and dried over magnesium sulfate, filtered and concentrated. This gave 1.35 g (98% of theory) of the title compound.

¹H-NMR (400 MHz, DMSO-d₆): δ=7.37 (d, 1H), 8.12 (dd, 1H), 8.16 (dd, 1H), 11.3 (s, 1H), 13.7 (s, 1H).

### Example 25A

### 5-Fluoro-7-nitro-1H-indole

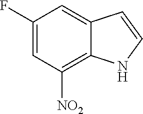

1.35 g (6.02 mmol) of the compound from example 24A were initially charged in 13.5 ml of quinoline, admixed with 349 mg (1.51 mmol) of copper chromium oxide and stirred at 205° C. for 2 h. After cooling, ethyl acetate was added and the mixture was extracted with 1 M hydrochloric acid. The organic phase was washed with saturated sodium chloride solution, dried over magnesium sulfate, filtered and concentrated. The residue was purified by means of flash chromatography (eluent: cyclohexane/dichloromethane 2:1) to obtain 975 mg (90% of theory) of the title compound.

¹H-NMR (400 MHz, DMSO-d₆): δ=6.74 (d, 1H), 7.63 (d, 1H), 7.92-8.01 (m, 2H), 12.0 (s, 1H).

### Example 26A

### 3-(5-Fluoro-7-nitro-1H-indol-3-yl)-3-[4-(trifluoromethyl)phenyl]propanoic acid

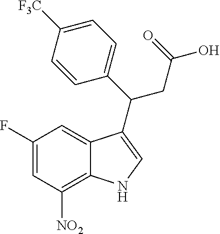

1.02 g (5.66 mmol) of the compound from example 25A were initially charged in 20 ml of acetonitrile, admixed with 5.10 g (17.0 mmol) of 2,2-dimethyl-5-[4-(trifluoromethyl)benzylidene]-1,3-dioxane-4,6-dione and heated under reflux for two days. The mixture was then concentrated and the residue was purified first by means of flash chromatography (eluent: dichloromethane/methanol 20:1) and then by means of preparative HPLC (RP18 column; eluent: acetonitrile/water gradient with addition of 1% formic acid). 1.33 g (54% of theory) of the title compound were obtained.

¹H-NMR (400 MHz, DMSO-d₆): δ=3.09 (dd, 1H), 3.20 (dd, 1H), 4.81 (t, 1H), 7.60-7.71 (m, 5H), 7.89-7.95 (m, 2H), 11.9 (s, 1H), 12.2 (s, 1H).

LC-MS (method 9): Rₜ=2.65 min; MS (ESIpos): m/z=397 [M+H]⁺.

### Example 27A

### Ethyl 3-(5-fluoro-7-nitro-1H-indol-3-yl)-3-[4-(trifluoromethyl)phenyl]propanoate

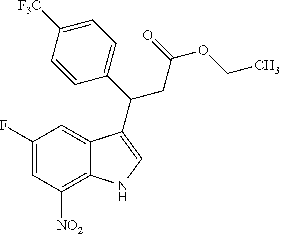

1.07 g (2.70 mmol) of the compound from example 26A were initially charged in 20 ml of diethyl ether, admixed with 842 mg (4.05 mmol) of thionyl chloride and stirred at RT for 1 h. Subsequently, 10 ml of ethanol were added and the reaction mixture was stirred at RT overnight. The mixture was then poured onto water and extracted with ethyl acetate. The organic phase was washed with saturated sodium chloride solution, dried over magnesium sulfate, filtered and concentrated. The residue was purified by means of flash chromatography (eluent: dichloromethane/cyclohexane 2:1) to obtain 560 mg (49% of theory) of the title compound.

¹H-NMR (400 MHz, DMSO-d₆): δ=1.03 (t, 3H), 3.20 (dd, 1H), 3.29 (dd, 1H), 3.96 (q, 2H), 4.84 (t, 1H), 7.60-7.63 (m, 2H), 7.66-7.70 (m, 2H), 7.72 (s, 1H), 7.91 (dd, 1H), 7.95 (dd, 1H), 11.9 (s, 1H).

MS (ESIneg): m/z=423 [M−H]⁻.

### WORKING EXAMPLES

### Example 1

### 3-(7-Ethyl-1H-indol-3-yl)-3-[4-(trifluoromethyl)phenyl]propan-1-ol

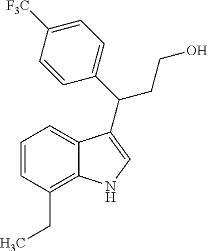

To 3.51 g of lithium aluminum hydride (92.4 mmol) in 150 ml of diethyl ether were slowly added, at 0° C., 12.0 g of the compound from example 2A (30.8 mmol). The mixture was stirred at RT overnight and the reaction was ended by adding 10 ml of isopropanol at 0° C. The reaction solution was neutralized with sat. aq. ammonium chloride solution. The aqueous phase was extracted with 200 ml of diethyl ether and the organic phase was washed with 50 ml of 1 N hydrochloric acid. The combined organic phases were dried over magnesium sulfate and freed of the solvent under reduced pressure. The residue obtained was 10.1 g (95% of theory) of the target compound.

¹H-NMR (400 MHz, CDCl₃): δ=1.35 (t, 3H), 2.22-2.33+2.44-2.54 (AB-Signal, 2m, 2H), 2.85 (q, 2H), 3.59-3.74 (m, 2H), 4.48 (t, 1H), 6.96-7.04 (m, 2H), 7.10 (d, 1H), 7.27 (m, 1H), 7.41 (d, 2H), 7.51 (d, 2H), 8.02 (s, 1H).

### Example 2

### 4-(7-Ethyl-1H-indol-3-yl)-4-[4-(trifluoromethyl)phenyl]butanenitrile

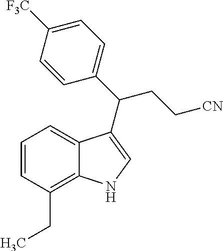

To 1.45 g of the compound from example 3A (3.41 mmol) in 14.5 ml of DMF were added 444 mg of potassium cyanide (6.82 mmol). The mixture was heated to 80° C. for 3 h and then the reaction was ended by adding 20 ml each of ethyl acetate and water. The organic phase was washed with 30 ml of sat. aq. sodium hydrogencarbonate solution, dried over magnesium sulfate and freed of the solvent under reduced pressure. The residue was purified by chromatography using a silica gel column (eluent: dichloromethane/cyclohexane 2:1). 960 mg (78% of theory) of the target compound were obtained.

¹H-NMR (400 MHz, DMSO-d₆): δ=1.23 (t, 3H), 2.31-2.39 (m, 1H), 2.40-2.45 (m, 2H), 2.82 (q, 2H), 4.33 (t, 1H), 6.82-6.89 (m, 2H), 7.22 (dd, 1H), 7.40 (d, 1H), 7.58 (d, 2H), 7.63 (d, 2H), 11.00 (s, 1H).

HPLC (method 4): Rₜ=5.15 min; MS (ESIneg): m/z=355.2 [M−H]⁻.

The enantiomers were separated by preparative HPLC on a chiral phase [column: Daicel Chiralcel OD-H, 5 μm, 250 mm×20 mm; eluent: isohexane/isopropanol 3:1; flow rate: 15 ml/min; temperature: 40° C.; UV detection: 220 nm]:

### Enantiomer 2-1:

Rₜ=6.43 min [column: Daicel Chiralcel OD-H, 5 um, 250 mm×4.6 mm; eluent: isohexane/isopropanol 3:1; flow rate: 1.0 ml/min; temperature: 25° C.; UV detection: 210 nm];

### Enantiomer 2-2:

Rₜ=8.40 min [column: Daicel Chiralcel OD-H, 5 μm, 250 mm×4.6 mm; eluent: isohexane/isopropanol 3:1; flow rate: 1.0 ml/min; temperature: 25° C.; UV detection: 210 nm].

### Example 3

### 4-(7-Ethyl-1H-indol-3-yl)-4-[4-(trifluoromethyl)phenyl]butan-1-ol

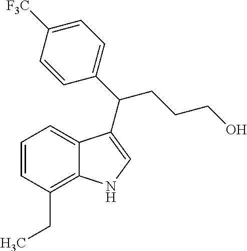

To 9.78 g of the compound from example 4A (26.0 mmol) in 100 ml of THF were added 2.47 g of lithium aluminum hydride (65.0 mmol) in 30 ml of THF, and the reaction mixture was stirred at 60° C. overnight. After cooling, first 100 ml of isopropanol, then 100 ml of 1 N hydrochloric acid were added. After the mixture had been filtered through a silica gel frit and washed through with ethyl acetate, the phases of the filtrate were separated. The aqueous phase was extracted with 100 ml of ethyl acetate. The organic phase was washed with 100 ml each of water, sat. aq. sodium hydrogencarbonate solution and sat. aq. sodium chloride solution. The combined organic phases were dried over magnesium sulfate and freed of the solvent under reduced pressure. The residue was purified by chromatography using a silica gel column (eluent: dichloromethane). 9.05 g (91% of theory) of the title compound were obtained.

¹H NMR (400 MHz, DMSO-d₆): δ=1.23 (t, 3H), 1.28-1.40+1.40-1.52 (AB signal, 2m, 2H), 1.97-2.07+2.12-2.23 (AB signal, 2m, 2H), 2.81 (q, 2H), 3.24 (td, 1H), 4.22 (t, 1H), 4.37 (t, 1H), 6.79-6.86 (m, 2H), 7.20 (dd, 1H), 7.29 (d, 1H), 7.54 (d, 2H), 7.60 (d, 2H), 10.89 (s, 1H).

HPLC (method 5): Rₜ=4.82 min; MS (ESIpos): m/z=362.3 [M+H]⁺.

The enantiomers were separated by preparative HPLC on a chiral phase [column: Daicel Chiralpak OD-H, 250 mm×20 mm; eluent: isopropanol/isohexane 20:80; flow rate: 20 ml/min; temperature: 24° C.; UV detection: 230 nm]:

### Enantiomer 3-1:

Rₜ=6.27 min [column: Daicel Chiralpak OD-H, 250 mm×4 mm; eluent: isopropanol/isohexane 20:80; flow rate: 1 ml/min; UV detection: 230 nm];

### Enantiomer 3-2:

Rₜ=8.67 min [column: Daicel Chiralpak OD-H, 250 mm×4 mm; eluent: isopropanol/isohexane 20:80; flow rate: 1 ml/min; UV detection: 230 nm].

### Example 4

### 5-(7-Ethyl-1H-indol-3-yl)-5-[4-(trifluoromethyl)phenyl]pentanenitrile

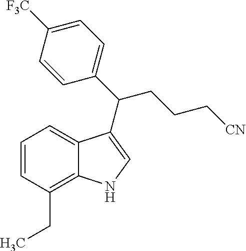

To 2.20 g of the compound from example 3 (5.26 mmol) in 28 ml of dichloromethane were added 1.34 ml of triethylamine (974 mg, 9.62 mmol) and 69 mg of 4-N,N-dimethylaminopyridine (0.57 mmol). The mixture was left to stir for 10 min and then 657 μl of methanesulfonyl chloride (973 mg, 8.49 mmol) were added at 0° C. After stirring at RT for 40 min, the mixture was diluted with 100 ml of diethyl ether. The mixture was extracted successively with 20 ml each of water, 1 N hydrochloric acid, water, sat. aq. sodium hydrogencarbonate solution, water and sat. aq. sodium chloride solution. The organic phase was dried over magnesium sulfate and freed of the solvent under reduced pressure.

The residue was dissolved in 28 ml of DMF, 728 mg of potassium cyanide (11.2 mmol) were added and the mixture was stirred at 80° C. overnight. After cooling, 30 ml each of water and diethyl ether were added. The organic phase was washed twice with 20 ml each of water and sat. aq. sodium chloride solution, dried over magnesium sulfate and freed of the solvent under reduced pressure. The residue was recrystallized from ethanol. 1.25 g (60% of theory) of the title compound were obtained.

¹H NMR (400 MHz, CDCl₃): δ=1.36 (t, 3H), 1.60-1.82 (m, 2H), 2.13-2.24+2.32-2.45 (AB signal, 2m, 2H), 2.37 (t, 2H), 2.85 (q, 2H), 4.25 (t, 1H), 6.97-7.04 (m, 2H), 7.10 (d, 1H), 7.24 (dd, 1H), 7.41 (d, 2H), 7.53 (d, 2H), 8.04 (s, 1H).

MS (CIpos): m/z=388.0 [M+NH₄]⁺.

The enantiomers were separated by preparative HPLC on a chiral phase [column: Daicel Chiralcel OD-H, 5 μm, 250 mm×20 mm; eluent: isohexane/isopropanol 4:1; flow rate: 15 ml/min; temperature: 24° C.; UV detection: 240 nm]:

### Enantiomer 4-1:

Rₜ=4.19 min [column: Daicel Chiralcel AD-H, 250 mm×4.6 mm; eluent: isopropanol/isohexane 30:70; flow rate: 1 ml/min; UV detection: 220 nm];

### Enantiomer 4-2:

Rₜ=4.80 min [column: Daicel Chiralcel AD-H, 250 mm×4.6 mm; eluent: isopropanol/isohexane 30:70; flow rate: 1 ml/min; UV detection: 220 nm].

### Example 5

### 3-(7-Methyl-1H-indol-3-yl)-3-[4-(trifluoromethyl)phenyl]propan-1-ol

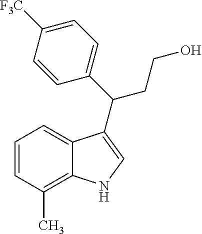

The title compound was prepared proceeding from 7-methylindole analogously to the synthesis of the compound from example 1.

¹H NMR (400 MHz, CDCl₃): δ=1.31 (t, 1H), 2.22-2.32+2.44-2.54 (AB signal, 2m, 2H), 2.61 (s, 3H), 3.67 (m_{c}, 2H), 4.48 (t, 1H), 6.93-7.00 (m, 2H), 7.11 (d, 1H), 7.26 (d, 1H), 7.43 (d, 2H), 7.51 (d, 2H), 7.99 (s, 1H).

MS (CIpos): m/z=334.3 [M+H]⁺.

### Example 6

### 4-(7-Methyl-1H-indol-3-yl)-4-[4-(trifluoromethyl)phenyl]butanenitrile

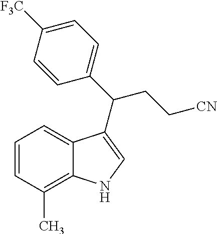

The title compound was prepared proceeding from the compound from example 5 analogously to the synthesis of the compound from example 2.

¹H NMR (400 MHz, CDCl₃): δ=2.33 (t, 2H), 2.33-2.43+2.54-2.62 (AB signal, 2m, 2H), 2.49 (s, 3H), 4.41 (t, 1H), 6.95-7.02 (m, 2H), 7.11 (d, 2H), 7.25 (d, 1H), 7.44 (d, 2H), 7.55 (d, 2H), 8.05 (s, 1H).

MS (CIpos): m/z=360.4 [M+NH₄]⁺.

The enantiomers were separated by preparative HPLC on a chiral phase [column: Daicel Chiralcel OD-H, 5 μm, 250 mm×20 mm; eluent: isohexane/isopropanol 3:1; flow rate: 15 ml/min; temperature: 40° C.; UV detection: 220 nm]:

### Enantiomer 6-1:

Rₜ=6.40 min [column: Daicel Chiralcel OD-H, 5 μm, 250 mm×4.6 mm; eluent: isohexane/isopropanol 3:1; flow rate: 1.0 ml/min; temperature: 25° C.; UV detection: 210 nm];

### Enantiomer 6-2:

Rₜ=8.47 min [column: Daicel Chiralcel OD-H, 5 μm, 250 mm×4.6 mm; eluent: isohexane/isopropanol 3:1; flow rate: 1.0 ml/min; temperature: 25° C.; UV detection: 210 nm].

### Example 7

### 4-(7-Methyl-1H-indol-3-yl)-4-[4-(trifluoromethyl)phenyl]butan-1-ol

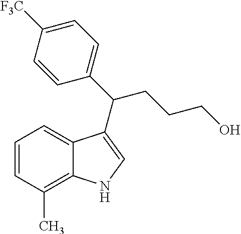

The title compound was prepared proceeding from the compound from example 6 analogously to the synthesis of the compound from example 3.

¹H NMR (400 MHz, CDCl₃): δ=1.21 (br. s, 1H), 1.50-1.72 (m, 2H), 2.04-2.16+2.25-2.36 (AB signal, 2m, 2H), 2.47 (s, 3H), 3.68 (td, 2H), 4.24 (t, 1H), 2.92-2.99 (m, 2H), 7.10 (d, 1H), 7.24 (d, 1H), 7.41 (d, 2H), 7.51 (d, 2H), 7.96 (s, 1H).

### Example 8

### 5-(7-Methyl-1H-indol-3-yl)-5-[4-(trifluoromethyl)phenyl]pentanenitrile

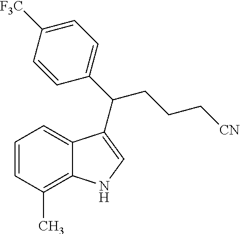

The title compound was prepared proceeding from the compound from example 7 analogously to the synthesis of the compound from example 4.

¹H NMR (400 MHz, CDCl₃): δ=1.59-1.82 (m, 2H), 2.12-2.24+2.31-2.43 (AB signal, 2m, 2H), 2.36 (t, 2H), 2.48 (s, 3H), 4.25 (t, 1H), 6.93-7.00 (m, 2H), 7.11 (d, 1H), 7.23 (d, 1H), 7.43 (d, 2H), 7.52 (d, 2H), 8.01 (s, 1H).

MS (CIpos): m/z=374.6 [M+NH₄]⁺.

The enantiomers were separated by preparative HPLC on a chiral phase [column: Daicel Chiralcel OD-H, 5 μm, 250 mm×20 mm; eluent: isohexane/isopropanol 3:1; flow rate: 15 ml/min; temperature: 24° C.; UV detection: 240 nm]:

### Enantiomer 8-1:

Rₜ=4.53 min [column: Daicel Chiralcel OD-H, 250 mm×4.6 mm; eluent: isopropanol/isohexane 50:50; flow rate: 1 ml/min; UV detection: 220 nm];

### Enantiomer 8-2:

Rₜ=5.76 min [column: Daicel Chiralcel OD-H, 250 mm×4.6 mm; eluent: isopropanol/isohexane 50:50; flow rate: 1 ml/min; UV detection: 220 nm].

### Example 9

### 3-(7-Bromo-1H-indol-3-yl)-3-[4-(trifluoromethyl)phenyl]propan-1-ol

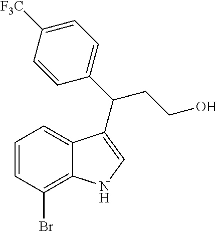

The title compound was prepared proceeding from 7-bromoindole analogously to the synthesis of the compound from example 1.

¹H NMR (400 MHz, CDCl₃): δ=1.29 (t, 1H), 2.22-2.32+2.43-2.53 (2m, AB signal, 2H), 3.59-3.74 (m, 2H), 4.48 (t, 1H), 6.91 (dd, 1H), 7.18 (d, 1H), 7.32 (d, 1H), 7.33 (d, 1H), 7.41 (d, 2H), 7.54 (d, 2H), 8.24 (s, 1H).

### Example 10

### 4-(7-Bromo-1H-indol-3-yl)-4-[4-(trifluoromethyl)phenyl]butanenitrile

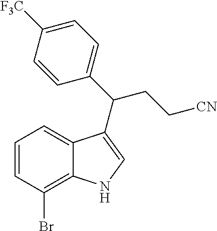

The title compound was prepared proceeding from the compound from example 9 analogously to the synthesis of the compound from example 2.

¹H NMR (400 MHz, CDCl₃): δ=2.26-2.45 (m, 3H), 2.50-2.61 (m, 1H), 4.40 (t, 1H), 6.94 (dd, 1H), 7.18 (d, 1H), 7.31 (d, 1H), 7.35 (d, 1H), 7.42 (d, 2H), 7.57 (d, 2H), 8.30 (s, 1H).

LC-MS (method 1): Rₜ=2.71 min; MS (ESIpos): m/z=407.1 [M+H]⁺.

### Example 11

### 4-(7-Bromo-1H-indol-3-yl)-4-[4-(trifluoromethyl)phenyl]butan-1-ol

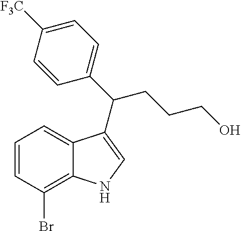

The title compound was prepared proceeding from the compound from example 10 analogously to the synthesis of the compound from example 3.

¹H NMR (400 MHz, CDCl₃): δ=1.23 (br. s, 1H), 1.50-1.70 (m, 2H), 2.05-2.16+2.25-2.35 (AB signal, 2m, 2H), 3.68 (t, 2H), 4.22 (t, 1H), 6.90 (dd, 1H), 7.18 (d, 1H), 7.30 (d, 1H), 7.31 (d, 1H), 7.39 (d, 1H), 7.52 (d, 2H), 8.22 (s, 1H).

### Example 12

### 5-(7-Bromo-1H-indol-3-yl)-5-[4-(trifluoromethyl)phenyl]pentanenitrile

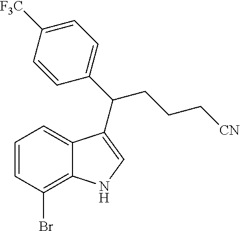

The title compound was prepared proceeding from the compound from example 11 analogously to the synthesis of the compound from example 4.

¹H NMR (400 MHz, CDCl₃): δ=1.61-1.80 (m, 2H), 2.14-2.23+2.32-2.43 (AB signal, 2m, 2H), 2.38 (m, 2H), 4.23 (t, 1H), 6.92 (dd, 1H), 7.19 (d, 1H), 7.30 (d, 1H), 7.33 (d, 1H), 7.39 (d, 2H), 7.54 (d, 2H), 8.26 (s, 1H).

MS (CIpos): m/z=438.0 [M+NH₄]⁺.

### Example 13

### 3-{3-Cyano-1-[4-(trifluoromethyl)phenyl]propyl}-1H-indole-7-carbonitrile

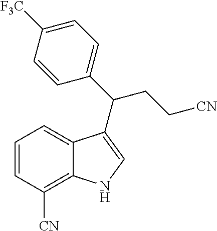

Argon gas was allowed to bubble through a solution of 50.0 mg of the compound from example 10 (0.123 mmol) in 1.2 ml of dry DMF for 10 min, and then 15.9 g of zinc cyanide (0.135 mmol) and 14 mg of tetrakis(triphenylphosphine)palladium (0.012 mmol) were added. The mixture was stirred in a microwave reactor at RT for 30 seconds and then at 200° C. for 15 min. After cooling, the mixture was filtered through kieselguhr and washed through with DMF. The solvent of the filtrate was removed under reduced pressure. 10 ml each of water and methyl tert-butyl ether were added to the residue. The organic phase was washed with 10 ml each of water and sat. aq. sodium chloride solution, filtered through Extrelut and freed of the solvent under reduced pressure. The residue was purified by means of preparative HPLC (eluent: acetonitrile/water with 0.1% formic acid, gradient 10:90→95:5). This gave 33 mg (76% of theory) of the title compound.

¹H NMR (400 MHz, CDCl₃): δ=2.25-2.44 (m, 3H), 2.51-2.62 (m, 1H), 4.44 (t, 1H), 7.11 (d, 1H), 7.25 (d, 1H), 7.42 (d, 2H), 7.52 (d, 1H), 7.59 (d, 2H), 8.73 (s, 1H).

MS (CIpos): m/z=371.1 [M+NH₄]⁺.

The enantiomers were separated by preparative HPLC on a chiral phase [column: Daicel Chiralcel OD-H, 5 μm, 250 mm×20 mm; eluent: isohexane/isopropanol 70:30; flow rate: 20 ml/min; temperature: 24° C.; UV detection: 230 nm]:

### Enantiomer 13-1:

Rₜ=5.94 min [column: Daicel Chiralcel OD-H, 250 mm×4 mm; eluent: isopropanol/isohexane 30:70; flow rate: 1 ml/min; UV detection: 225 nm];

### Enantiomer 13-2:

Rₜ=10.04 min [column: Daicel Chiralcel OD-H, 250 mm×4 mm; eluent: isopropanol/isohexane 30:70; flow rate: 1 ml/min; UV detection: 225 nm].

### Example 14

### 3-{4-Cyano-1-[4-(trifluoromethyl)phenyl]butyl}-1H-indole-7-carbonitrile

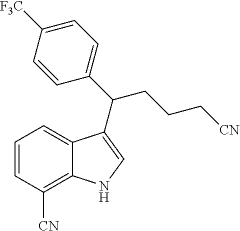

Argon gas was allowed to bubble through a solution of 55.0 mg of the compound from example 12 (0.131 mmol) in 1.3 ml of dry DMF for 10 min, and then 16.9 g of zinc cyanide (0.144 mmol) and 15 mg of tetrakis(triphenylphosphine)palladium (0.013 mmol) were added. The mixture was stirred in a microwave reactor at RT for 30 seconds and then at 200° C. for 15 min. After cooling, the mixture was filtered through kieselguhr and washed through with DMF. The solvent of the filtrate was removed under reduced pressure. 10 ml each of water and methyl tert-butyl ether were added to the residue. The organic phase was washed with 10 ml each of water and sat. aq. sodium chloride solution, filtered through Extrelut and freed of the solvent under reduced pressure. The residue was purified by means of preparative HPLC (eluent: acetonitrile/water with 0.1% formic acid, gradient 30:70→95:5). This gave 36 mg (75% of theory) of the title compound.

¹H NMR (400 MHz, CDCl₃): δ=1.59-1.80 (m, 2H), 2.14-2.25 (m, 1H), 2.30-2.46 (m, 2H), 4.26 (t, 2H), 7.08 (dd, 1H), 7.38 (d, 2H), 7.50 (d, 1H), 7.56 (d, 2H), 7.57 (d, 1H), 8.66 (s, 1H).

MS (ESIneg): m/z=366.1 [M−H]⁻.

### Example 15

### 3-(7-Amino-1H-indol-3-yl)-3-[4-(trifluoromethyl)phenyl]propan-1-ol

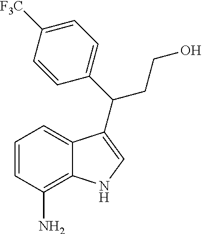

To 61.8 mg of lithium aluminum hydride (1.63 mmol) in 5 ml of THF were slowly added 175 mg of the compound from example 6A (0.465 mmol). The mixture was stirred at 60° C. overnight and the reaction was ended by adding 20 ml of water. The mixture was filtered through Celite and washed through with ethyl acetate and water. The filtrate was extracted with 25 ml of ethyl acetate. The organic phase was dried over magnesium sulfate and freed of the solvent under reduced pressure. The residue was purified by means of preparative HPLC (eluent: acetonitrile/water with 0.1% formic acid, gradient 10:90→95:5). This gave 112 mg (72% of theory) of the title compound.

¹H NMR (400 MHz, DMSO-d₆): δ=2.05-2.16+2.25-2.38 (AB signal, 2m, 2H), 3.30-3.42 (m, 2H), 4.34 (t, 1H), 4.48 (t, 1H), 4.96 (s, 2H), 6.22-6.28 (m, 1H), 6.58-6.62 (m, 2H), 7.24 (d, 1H), 7.50 (d, 2H), 7.59 (d, 2H), 10.47 (s, 1H).

HPLC (method 5): Rₜ=3.81 min; MS (ESIneg): m/z=333.1 [M−H]⁻.

### Example 16

### N-(3-{3-Hydroxy-1-[4-(trifluoromethyl)phenyl]propyl}-1H-indol-7-yl)methanesulfonamide

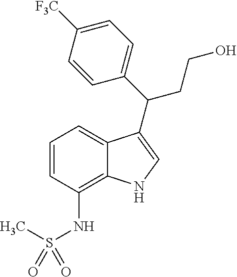

50.0 mg of the compound from example 15 (0.150 mmol) were initially charged in 1 ml of THF. At 0° C., 13 μl of pyridine (13 mg, 0.17 mmol) and, 5 min thereafter, 13 μl of methanesulfonyl chloride (19 mg, 0.17 mmol) were added. The mixture was subsequently left to stir at RT overnight. The solvent was then removed under reduced pressure and the residue was purified by means of preparative HPLC (eluent: acetonitrile/water with 0.1% formic acid, gradient 10:90→95:5). This gave 53.5 mg (87% of theory) of the target compound.

¹H NMR (400 MHz, DMSO-d₆): δ=2.08-2.19+2.27-2.38 (AB signal, 2m, 2H), 2.96 (s, 3H), 3.29-3.42 (m, 2H), 4.43 (t, 1H), 4.51 (t, 1H), 6.69 (dd, 1H), 7.01 (d, 1H), 7.24 (d, 1H), 7.35 (d, 1H), 7.54 (d, 2H), 7.61 (d, 2H), 9.27 (s, 1H), 10.69 (s, 1H).

HPLC (method 5): Rₜ=4.07 min; MS (CIpos): m/z=430.1 [M+NH₄]⁺.

### Example 17

### N-(3-{4-Cyano-1-[4-(trifluoromethyl)phenyl]butyl}-1H-indol-7-yl)methanesulfonamide

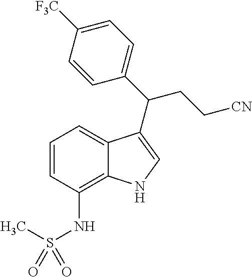

To 37.8 mg of the compound from example 7A (0.0771 mmol) in 1 ml of DMF were added 10.0 mg of potassium cyanide (0.154 mmol), and the mixture was stirred at 80° C. for 3 h. Then 5 ml of ethyl acetate were added. The mixture was extracted with 5 ml each of water and sat. aq. sodium hydrogencarbonate solution. The organic phase was dried over magnesium sulfate and freed of the solvent under reduced pressure. The residue was purified by means of preparative HPLC (eluent: acetonitrile/water with 0.1% formic acid, gradient 10:90→95:5). This gave 24 mg (73% of theory) of the target compound.

¹H NMR (400 MHz, DMSO-d₆): δ=2.30-2.40 (m, 2H), 2.43 (t, 2H), 2.96 (s, 3H), 4.35 (t, 1H), 6.92 (dd, 1H), 7.03 (d, 1H), 7.28 (d, 1H), 7.44 (d, 1H), 7.59 (d, 2H), 7.64 (d, 2H), 9.29 (s, 1H), 10.80 (s, 1H).

HPLC (method 5): Rₜ=4.57 min; MS (ESIneg): m/z=420.1 [M−H]⁻.

The enantiomers were separated by preparative HPLC on a chiral phase [column: Daicel Chiralpak OD-H, 5 μm 250 mm×20 mm, eluent: isopropanol/isohexane 50:50; flow rate: 20 ml/min; temperature: 25° C.; UV detection: 230 nm]:

### Enantiomer 17-1:

Rₜ=10.70 min [column: Daicel Chiralpak OD-H, 250 mm×4 mm; eluent: isopropanol/isohexane 50:50; flow rate: 1 ml/min; UV detection: 230 nm];

### Enantiomer 17-2:

Rₜ=12.47 min [column: Daicel Chiralpak OD-H, 250 mm×4 mm; eluent: isopropanol/isohexane 50:50; flow rate: 1 ml/min; UV detection: 230 nm].

### Example 18

### N-(3-{4-Hydroxy-1-[4-(trifluoromethyl)phenyl]butyl}-1H-indol-7-yl)methanesulfonamide

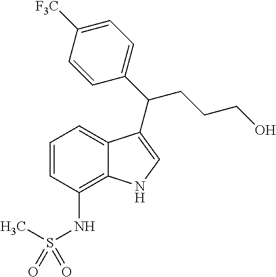

The title compound was prepared proceeding from the compound from example 17 analogously to the synthesis of the compound from example 3.

¹H NMR (400 MHz, DMSO-d₆): δ=1.28-1.39+1.39-1.51 (AB signal, 2m, 2H), 1.96-2.07+2.12-2.22 (AB signal, 2m, 2H), 2.96 (s, 3H), 3.42 (td, 2H), 4.25 (t, 1H), 4.38 (t, 1H), 6.89 (dd, 1H), 7.01 (d, 1H), 7.25 (d, 1H), 7.34 (d, 1H), 7.55 (d, 2H), 7.61 (d, 2H), 9.27 (s, 1H), 10.70 (s, 1H).

HPLC (method 5): Rₜ=4.28 min; MS (ESIneg): m/z=425.1 [M−H]⁻.

### Example 19

### N-(3-{4-Cyano-1-[4-(trifluoromethyl)phenyl]butyl}-1H-indol-7-yl)methanesulfonamide

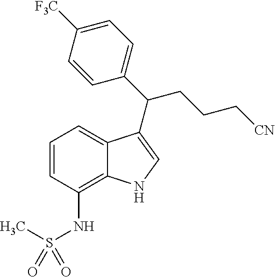

The title compound was prepared proceeding from the compound from example 18 analogously to the synthesis of the compound from example 4.

¹H NMR (400 MHz, DMSO-d₆): δ=1.41-1.64 (m, 2H), 2.05-2.16+2.19-2.30 (AB signal, 2m, 2H), 2.55 (t, 2H), 2.96 (s, 3H), 4.33 (t, 1H), 6.90 (dd, 1H), 7.02 (d, 1H), 7.27 (d, 1H), 7.38 (d, 1H), 7.57 (d, 2H), 7.63 (d, 2H), 9.28 (s, 1H), 10.74 (s, 1H).

LC-MS (method 2): Rₜ=3.53 min; MS (ESIpos): m/z=436.1 [M+H]⁺.

### Example 20

### 3-(7-Ethyl-1H-indol-3-yl)-3-(4-fluorophenyl)propan-1-ol

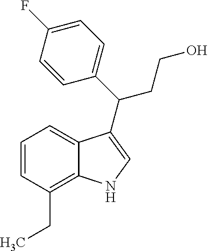

The title compound was prepared proceeding from 4-fluorobenzaldehyde analogously to the synthesis of the compound from example 1.

¹H NMR (400 MHz, DMSO-d₆): δ=1.23 (t, 3H), 2.03-2.14+2.24-2.34 (2m, AB signal, 2H), 2.81 (q, 2H), 3.30-3.40 (m, 2H), 4.30 (t, 1H), 4.45 (t, 1H), 6.78-6.86 (m, 2H), 7.02-7.07 (m, 2H), 7.18 (d, 1H), 7.23 (d, 1H), 7.30-7.35 (m, 2H), 10.82 (s, 1H).

HPLC (method 5): Rₜ=4.44 min; MS (CIpos): m/z=315.1 [M+NH₄]⁺.

### Example 21

### 4-(7-Ethyl-1H-indol-3-yl)-4-(4-fluorophenyl)butanenitrile

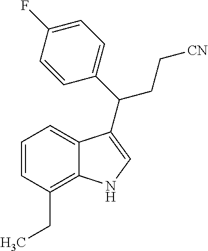

The title compound was prepared proceeding from the compound from example 20 analogously to the synthesis of compound 2.

¹H NMR (400 MHz, DMSO-d₆): δ=1.23 (t, 3H), 2.24-2.55 (m, 4H), 2.81 (q, 2H), 4.22 (t, 1H), 6.81-6.88 (m, 1H), 7.09 (m_{c}, 2H), 7.21 (dd, 1H), 7.32-7.40 (m, 3H), 10.94 (s, 1H).

HPLC (method 5): Rₜ=4.97 min; MS (ESIneg): m/z=305.2 [M−H]⁻.

### Example 22

### 3-[7-(Trifluoromethyl)-1H-indol-3-yl]-3-[4-(trifluoromethyl)phenyl]propan-1-ol

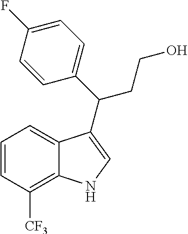

The title compound was prepared proceeding from 7-trifluoromethylindole [Y. Murakami et al., Chem. Pharm. Bull. 41 (11), 1910-1919 (1993)] analogously to the synthesis of the compound from example 1.

¹H NMR (400 MHz, DMSO-d₆): δ=2.12-2.21+2.30-2.40 (2m, AB signal, 2H), 3.30-3.43 (m, 2H), 4.51 (t, 1H), 4.53 (t, 1H), 7.07 (dd, 1H), 7.39 (d, 1H), 7.47 (d, 1H), 7.56 (d, 2H), 7.61 (d, 2H), 7.69 (d, 1H), 11.36 (s, 1H).

LC-MS (method 3): Rₜ=3.90 min; MS (ESIpos): m/z=388.2 [M+H]⁺.

### Example 23

### 4-[7-(Trifluoromethyl)-1H-indol-3-yl]-4-[4-(trifluoromethyl)phenyl]butanenitrile

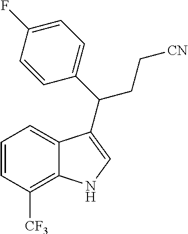

The title compound was prepared proceeding from the compound from example 22 analogously to the synthesis of the compound from example 2.

¹H NMR (400 MHz, DMSO-d₆): δ=2.31-2.60 (m, 4H), 4.42 (t, 1H), 7.09 (dd, 1H), 7.41 (d, 1H), 7.58 (d, 1H), 7.61 (d, 2H), 7.64 (d, 2H), 7.73 (d, 1H), 11.45 (s, 1H).

LC-MS (method 3): Rₜ=4.17 min; MS (CIpos): m/z=397.2 [M+NH₄]⁺.

### Example 24

### 3-(7-Methoxy-1H-indol-3-yl)-3-[4-(trifluoromethyl)phenyl]propan-1-ol

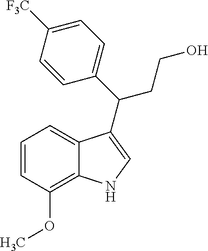

The title compound was prepared proceeding from 7-methoxyindole analogously to the synthesis of the compound from example 1.

¹H NMR (400 MHz, CDCl₃): δ=1.28 (t, 1H), 2.22-2.32+2.43-2.53 (2m, AB signal, 2H), 3.60-3.73 (m, 2H), 3.94 (s, 3H), 4.47 (t, 1H), 6.62 (d, 1H), 6.94 (dd, 1H), 7.01 (d, 1H), 7.07 (d, 1H), 7.43 (d, 2H), 7.51 (d, 2H), 8.26 (s, 1H).

### Example 25

### 4-(7-Methoxy-1H-indol-3-yl)-4-[4-(trifluoromethyl)phenyl]butanenitrile

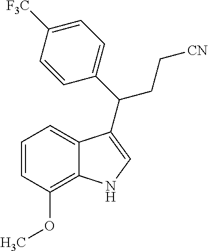

The title compound was prepared proceeding from the compound from example 24 analogously to the synthesis of the compound from example 2.

¹H NMR (400 MHz, CDCl₃): δ=2.30-2.43 (m, 3H), 2.51-2.63 (m, 1H), 3.95 (s, 3H), 4.39 (t, 1H), 6.64 (dd, 1H), 6.94-7.00 (m, 2H), 7.07 (d, 1H), 7.44 (d, 2H), 7.55 (d, 2H), 8.31 (s, 1H).

MS (CIpos): m/z=376.0 [M+NH₄]⁺.

The compounds listed in the table which follows were prepared analogously to the synthesis of the compound from example 1.

| Example | | Starting | |
|---|---|---|---|
| No. | Structure | compound | Analytical data |
| 26 | 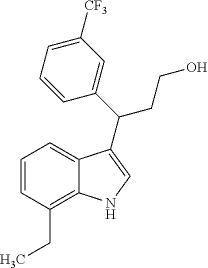 | 3-Trifluoro- methylbenz- aldehyde | LC-MS (method 6): Rₜ = 2.56 min; MS (ESIpos): m/z = 348.1 [M + H]⁺. |
| 27 | 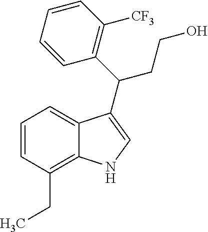 | 2-Trifluoro- methylbenz- aldehyde | LC-MS (method 2): Rₜ = 3.62 min; MS (ESIpos): m/z = 348.3 [M + H]⁺. |
| 28 | 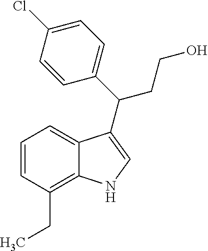 | 4-Chloro- benzalde- hyde | HPLC (method 5): Rₜ = 4.44 min; MS (CIpos): m/z = 314.0 [M + H]⁺. |
| 29 | 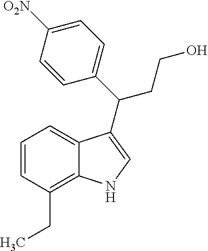 | 4-Nitro- benzalde- hyde | HPLC (method 5): Rₜ = 4.38 min; MS (ESIpos): m/z = 325.2 [M + H]⁺. |
| 30 | 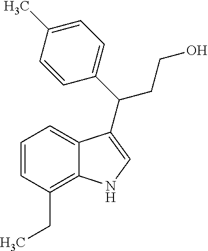 | 4-Methyl- benzalde- hyde | HPLC (method 5): Rₜ = 4.53 min; MS (ESIpos): m/z = 294.3 [M + H]⁺. |
| 31 | 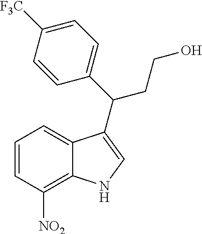 | 7-Nitro- indole* | HPLC (method 4): Rₜ = 4.51 min; MS (ESIneg): m/z = 363.2 [M − H]⁻ |

*the reduction of the ethyl carboxylate to the primary alcohol (cf. example 2A → example 1) was effected here with 2 eq. of lithium borohydride in THF (instead of lithium aluminum hydride in diethyl ether).

### Example 32

N-(3-{3-Cyano-3-fluoro-1-[4-(trifluoromethyl)phenyl]propyl}-1H-indol-7-yl)methanesulfonamide

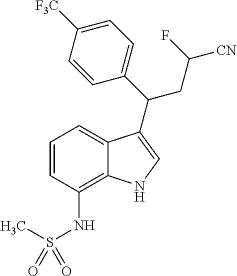

To 100 mg of the compound from example 8A (0.21 mmol, 87% purity) in 3 ml of ethyl acetate were added 33 mg of benzyltri-n-butylammonium chloride (0.11 mmol), 15 mg of potassium cyanide (0.22 mmol) and 6 ml of water. After stirring at RT for 1 h, 5 ml of ethyl acetate were added, and the solution was dried over sodium sulfate. The solids were filtered off and the solvent was removed under reduced pressure. The residue was dissolved in 2 ml of dichloromethane and admixed at 0° C. with 38 mg of diethylaminosulfur trifluoride (0.23 mmol), and the solution was subsequently stirred at RT for 16 h. After adding 1 ml of water, the crude product was first prepurified by means of preparative HPLC (eluent: acetonitrile/water, gradient 30:70→98:2). The solvent was removed from the product-containing fractions under reduced pressure, and the residue was purified further by column chromatography on silica gel (eluent: cyclohexane/ethyl acetate 1:1). This gave 9 mg (13% of theory) of the target compound.

¹H NMR (400 MHz, DMSO-d₆): δ=2.72-3.05 (m, 5H), 4.48-4.57 (m, 1H), 5.35-5.63 (m, 1H), 6.89-6.95 (m, 1H), 7.01-7.05 (m, 1H), 7.29-7.37 (m, 1H), 7.48-7.56 (dd, 1H), 7.62-7.66 (m, 4H), 9.28 (s, 1H), 10.83 (d, 1H).

LC-MS (method 8): Rₜ=1.30 min; MS (ESIpos): m/z=440.1 [M+H]⁺.

### Example 33

### 3-(7-Ethyl-1H-indol-3-yl)-3-naphth-2-ylpropan-1-ol

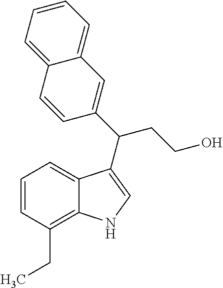

The title compound was prepared proceeding from 2-naphthaldehyde analogously to the synthesis of the compound from example 1.

¹H NMR (400 MHz, DMSO-d₆): δ=1.23 (t, 3H), 2.17-2.29 (m, 1H), 2.32-2.43 (m, 1H), 2.81 (q, 2H), 3.34-3.46 (m, 2H), 4.42-4.51 (m, 2H), 6.74-6.85 (m, 2H), 7.23 (d, 1H), 7.30 (s, 1H), 7.38-7.49 (m, 3H), 7.73-7.88 (m, 4H), 10.85 (s, 1H).

HPLC (method 5): Rₜ=4.76 min; MS (CIpos): m/z=347.2 [M+NH₄]⁺.

### Example 34

### 3-(7-Ethyl-1H-indol-3-yl)-3-naphth-2-ylbutanenitrile

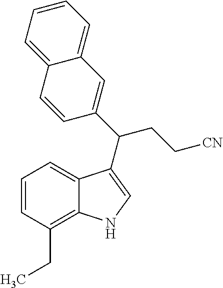

The title compound was prepared proceeding from the compound from example 33 analogously to the synthesis of the compound from example 2.

¹H NMR (400 MHz, DMSO-d₆): δ=1.23 (t, 3H), 2.40-2.52 (m, 4H), 2.81 (q, 2H), 4.35-4.41 (m, 1H), 6.78-6.87 (m, 2H), 7.25 (d, 1H), 7.38-7.51 (m, 4H), 7.77-7.93 (m, 4H), 10.95 (s, 1H). LC-MS (method 7): Rₜ=2.43 min; MS (ESIpos): m/z=339.3 [M+H]⁺.

### Example 35

### 3-(7-Bromo-1H-pyrrolo[2,3-c]pyridin-3-yl)-3-[4-(trifluoromethyl)phenyl]propan-1-ol

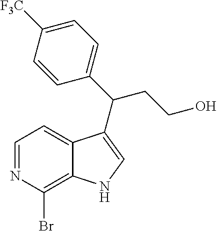

The title compound was prepared proceeding from 7-bromo-1H-pyrrolo[2,3-c]pyridine analogously to the synthesis of the compound from example 1.

¹H NMR (400 MHz, DMSO-d₆): δ=2.14-2.23 (m, 1H), 2.29-2.38 (m, 1H), 3.26-3.43 (m, 2H), 4.47 (t, 1H), 4.51 (t, 1H), 7.43 (d, 1H), 7.56 (d, 2H), 7.61 (d, 2H), 7.67 (d, 1H), 7.79 (d, 1H), 11.75 (s, 1H).

HPLC (method 4): Rₜ=3.82 min; MS (CIpos): m/z=399.0 [M+H]⁺.

### Example 36

### 3-(7-Bromo-1H-pyrrolo[2,3-c]pyridin-3-yl)-3-[4-(trifluoromethyl)phenyl]butanenitrile

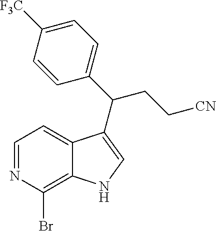

The title compound was prepared proceeding from the compound from example 35 analogously to the synthesis of the compound from example 2.

¹H NMR (400 MHz, DMSO-d₆): δ=2.34-2.59 (m, 4H), 4.35-4.42 (m, 1H), 7.48 (d, 1H), 7.61 (d, 2H), 7.65 (d, 2H), 7.79 (s, 1H), 7.81 (d, 1H), 11.87 (s, 1H).

HPLC (method 4): Rₜ=4.02 min; MS (CIpos): m/z=408.0 [M+H]⁺.

### Example 37

### N-(3-{1-[2-Fluoro-4-(trifluoromethyl)phenyl]-3-hydroxypropyl}-1H-indol-7-yl)-methanesulfonamide

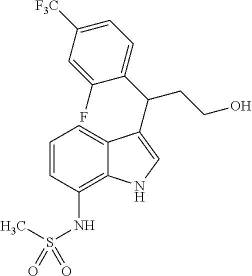

The title compound was prepared proceeding from 985 mg (2.09 mmol) of the compound from example 17A analogously to the synthesis of the compound from example 1. However, tetrahydrofuran was used as the solvent and the mixture was stirred at 60° C. for 2 h. The crude product was purified by means of preparative HPLC (RP18 column; eluent: acetonitrile/water gradient with addition of 0.1% formic acid) to obtain 630 mg (70% of theory) of the title compound.

¹H NMR (400 MHz, DMSO-d₆): δ=2.13-2.23 (m, 1H), 2.30-2.40 (m, 1H), 2.96 (s, 3H), 3.33-3.46 (m, 2H), 4.53 (t, 1H), 4.71 (t, 1H), 6.92 (t, 1H), 7.03 (d, 1H), 7.25 (d, 1H), 7.33 (d, 1H), 7.49 (d, 1H), 7.57-7.63 (m, 2H), 9.29 (s, 1H), 10.7 (s, 1H).

LC-MS (method 9): Rₜ=2.26 min; MS (ESIpos): m/z=431 [M+H]⁺.

The compounds listed in the table which follows were prepared analogously to the synthesis of the compound from example 37. Alternatively, it was also possible to stir at 60° C. only for 1 h and to effect the workup by quenching with water and 1 M hydrochloric acid, extracting the aqueous phase repeatedly with water, washing the organic phases with saturated aqueous sodium chloride solution, drying with sodium sulfate, filtering and concentrating.

| | | | Yield |
|---|---|---|---|
| Example | | Starting | (% of theory); analytical |
| No. | Structure | compound | data |
| 38 | 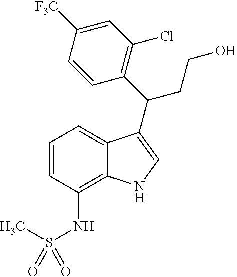 | 18A | 69% LC-MS (method 7): Rₜ = 1.89 min; MS (ESIpos): m/z = 447 [M + H]⁺. ¹H NMR (400 MHz, DMSO- d₆): δ = 2.09-2.19 (m, 1H), 2.27-2.38 (m, 1H), 2.97 (s, 3H), 3.36-3.47 (m, 2H), 4.54 (t, 1H), 4.87 (t, 1H), 6.92 (t, 1H), 7.03 (d, 1H), 7.22 (d, 1H), 7.35 (d, 1H), 7.56-7.63 (m, 2H), 7.82 (s, 1H), 9.30 (s, 1H), 10.8 (s, 1H). |
| 39 | 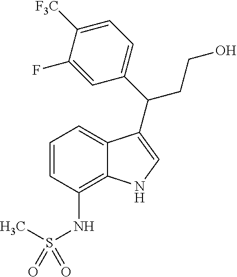 | 19A | 69% LC-MS (method 9): Rₜ = 2.20 min; MS (ESIpos): m/z = 431 [M + H]⁺ ¹H NMR (400 MHz, DMSO- d₆): δ = 2.11-2.22 (m, 1H), 2.26-2.38 (m, 1H), 2.97 (s, 3H), 3.29-3.43 (m, 2H), 4.46 (t, 1H), 4.54 (t, 1H), 6.91 (t, 1H), 7.03 (d, 1H), 7.30 (d, 1H), 7.36-7.41 (m, 2H), 7.47 (d, 1H), 7.65 (t, 1H), 9.28 (s, 1H), 10.7 (s, 1H). |
| 40 | 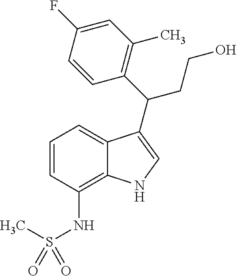 | 20A | 68% LC-MS (method 9): Rₜ = 2.06 min; MS (ESIpos): m/z = 377 [M + H]⁺ ¹H NMR (400 MHz, DMSO- d₆): δ = 1.96-2.06 (m, 1H), 2.20-2.30 (m, 1H), 2.41 (s, 3H), 2.96 (s, 3H), 3.29-3.45 (m, 2H), 4.47-4.54 (m, 2H), 6.86-6.92 (m, 2H), 6.96-7.03 (m, 2H), 7.14-7.22 (m, 3H), 9.27 (s, 1H), 10.6 (s, 1H). |
| 41 | 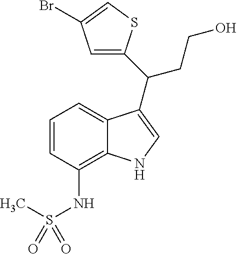 | 21A | 31% LC-MS (method 9): Rₜ = 2.04 min; MS (ESIneg): m/z = 427 [M − H]⁻ ¹H NMR (400 MHz, DMSO- d₆): δ = 2.12-2.33 (m, 2H), 2.98 (s, 3H), 3.39 (q, 2H), 4.54 (t, 1H), 4.61 (t, 1H), 6.94 (t, 1H), 6.99 (s, 1H), 7.04 (d, 1H), 7.28-7.34 (m, 2H), 7.39 (d, 1H), 9.30 (s, 1H), 10.7 (d, 1H). |
| 42 | 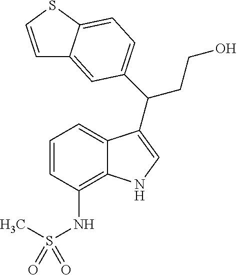 | 22A | 47% HPLC (method 5): Rₜ = 4.12 min; MS (CIpos): m/z = 401 [M + H]⁺ ¹H NMR (400 MHz, DMSO- d₆): δ = 2.12-2.23 (m, 1H), 2.29-2.39 (m, 1H), 2.95 (s, 3H), 3.31-3.44 (m, 2H), 4.42 (t, 1H), 4.46 (t, 1H), 6.85 (t, 1H), 6.99 (d, 1H), 7.25 (d, 1H), 7.30-7.35 (m, 2H), 7.38 (d, 1H), 7.69 (d, 1H), 7.81 (s, 1H), 7.84 (d, 1H), 9.26 (s, 1H), 10.7 (s, 1H). |
| 43 | 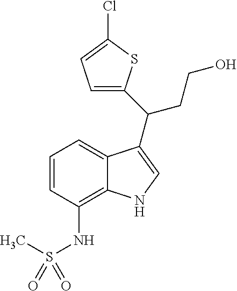 | 23A | 85% LC-MS (method 9): Rₜ = 2.03 min; MS (ESIpos): m/z = 385 [M + H]⁺ ¹H NMR (400 MHz, DMSO- d₆): δ = 2.09-2.30 (m, 2H), 2.98 (s, 3H), 3.40 (q, 2H), 4.50-4.58 (m, 2H), 6.86 (d, 1H), 6.89 (d, 1H), 6.93 (t, 1H), 7.04 (d, 1H), 7.27-7.33 (m, 2H), 9.30 (s, 1H), 10.7 (d, 1H). |

### Example 44

### N-(3-{3-Cyano-1-[2-fluoro-4-(trifluoromethyl)phenyl]propyl}-1H-indol-7-yl)methanesulfonamide

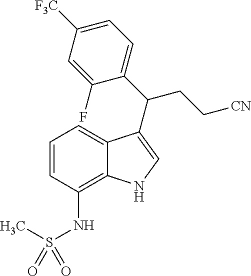

To 630 mg (1.46 mmol) of the compound from example 37 in 30 ml of dichloromethane were added 18 mg (0.15 mmol) of 4-N,N-dimethylaminopyridine and 0.35 ml (2.45 mmol) of triethylamine. The mixture was left to stir for 5 min and then 0.17 ml (2.20 mmol) of methanesulfonyl chloride were added. After stirring at RT overnight, dichloromethane was added and the solution was washed with 1 M hydrochloric acid, water and sat. sodium chloride solution. The organic phase was dried over magnesium sulfate, filtered and concentrated. The residue was purified by means of preparative HPLC (RP18 column; eluent: acetonitrile/water gradient with addition of 1% formic acid). The intermediate was dissolved in DMF, 160 mg (2.46 mmol) of potassium cyanide were added and the solution was stirred at 80° C. for 4 h. The reaction mixture was concentrated, and the residue was taken up in ethyl acetate and washed successively with sat. sodium hydrogencarbonate solution and sat. sodium chloride solution. The organic phase was dried over magnesium sulfate, filtered and concentrated, and the residue was purified by means of preparative HPLC (RP18 column; eluent: acetonitrile/water gradient with addition of 1% formic acid). This gave 439 mg (23% of theory) of the title compound.

¹H NMR (400 MHz, DMSO-d₆): δ=2.32-2.42 (m, 1H), 2.47-2.58 (m, 3H), 2.97 (s, 3H), 4.60-4.66 (m, 1H), 6.95 (t, 1H), 7.05 (d, 1H), 7.28 (d, 1H), 7.43 (d, 1H), 7.52 (d, 1H), 7.62-7.67 (m, 2H), 9.31 (s, 1H), 10.9 (s, 1H).

LC-MS (method 7): Rₜ=2.05 min; MS (ESIpos): m/z=440 [M+H]⁺.

The compounds listed in the table which follows were prepared analogously to the synthesis of the compound from example 44. Alternatively, it was also possible to purify the crude products by means of preparative HPLC without preceding workup:

| | | | Yield |
|---|---|---|---|
| Example | | Starting | (% of theory); analytical |
| No. | Structure | compound | data |
| 45 | 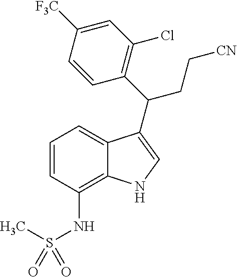 | 38 | 33% LC-MS (method 7): Rₜ = 2.14min; MS (ESIpos): m/z = 456 [M + H]⁺. ¹H NMR (400 MHz, DMSO- d₆): δ = 2.25-2.37 (m, 1H), 2.46-2.59 (m, 3H), 2.98 (s, 3H), 4.82(t, IH), 6.95 (t, 1H), 7.05 (d, 1H), 7.25 (d, 1H), 7.45 (d, 1H), 7.60-7.66 (m, 2H), 7.86 (s, 1H), 9.31 (s, 1H), 10.9 (s, 1H). |
| 46 | 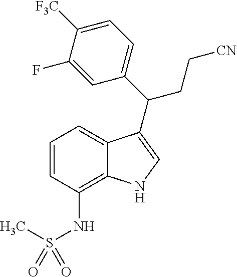 | 39 | 13% LC-MS (method 7): Rₜ = 2.04min; MS(ESIpos): m/z = 440 [M + H]⁺ ¹H NMR (400 MHz, DMSO- d₆): δ = 2.35-2.49 (m, 4H), 2.97 (s, 3H), 4.35-4.40 (m, 1H), 6.94 (d, 1H), 7.04 (d, 1H), 7.34 (d, 1H), 7.42 (d, 1H), 7.47 (d, 1H), 7.55 (d, 1H), 7.68 (t, 1H), 9.30 (s, 1H), 10.8 (s, 1H). |
| 47 | 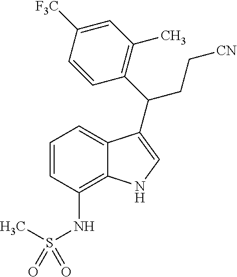 | 40 | 44% LC-MS (method 7): Rₜ = 1.87 min; MS (ESIpos): m/z = 386 [M + H]⁺ ¹H NMR (400 MHz, DMSO- d₆): δ = 2.15-2.26 (m, 1H), 2.41 (s, 3H), 2.36-2.49 (m, 3H), 2.97 (s, 3H), 4.43 (t, 1H), 6.90-6.97 (m, 2H), 6.99- 7.06 (m, 2H), 7.20-7.31 (m, 3H), 9.28 (s, 1H), 10.7 (s, 1H). |
| 48 | 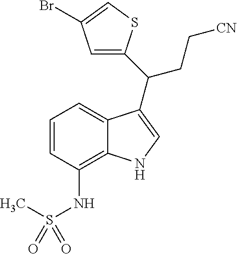 | 41 | 37% HPLC (method 5): Rₜ = 4.39 min; MS (Clpos): m/z = 455 [M + NH₄]⁺ ¹H NMR (400 MHz, DMSO- d₆): δ =2.31-2.53 (m, 4H), 2.98 (s, 3H), 4.48-4.55 (m, 1H), 6.96 (t, 1H), 7.06 (d, 1H), 7.10 (d, 1H), 7.33 (d, 1H), 7.40 (d, 1H), 7.44 (d, 1H), 9.31 (s, 1H), 10.8 (s, 1H). |
| 49 | 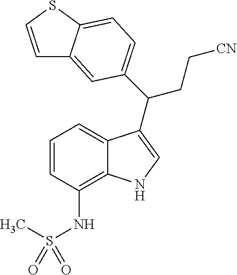 | 42 | 21% LC-MS (Methode 7): Rₜ = 1.95 min; MS (ESIpos): m/z = 410 [M + H]⁺ ¹H NMR (400 MHz, DMSO- d₆): δ = 2.31-2.56 (m, 4H), 2.95 (s, 3H), 4.31-4.37 (m, 1H), 6.88 (t, 1H), 7.01 (d, 1H), 7.28 (d, 1H), 7.36 (d, 1H), 7.38-7.44 (m, 2H), 7.72 (d, 1H), 7.84-7.91 (m, 2H), 9.27 (s, 1H), 10.7 (s, 1H). |
| 50* | 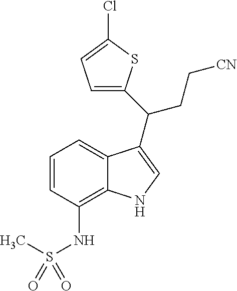 | 43 | 37% LC-MS (method 9): Rₜ = 2.28 min; MS (ESIpos): m/z = 394 [M + H]⁺ ¹H NMR (400 MHz, DMSO- d₆): δ = 2.29-2.57 (m, 4H), 2.71 (s, 3H), 4.43-4.50 (m, 1H), 6.91-6.98 (m, 3H), 7.06 (d, 1H), 7.29-7.33 (m, 1H), 7.38 (d, 1H), 9.32 (s, 1H), 10.79-10.84 (m, 1H). |

*a difference in the second part of the reaction (conversion of the mesylate to the cyanide) was that the mixture was stirred in DMF at 100° C. for 2 h.

### Example 51

### 3-(5-Fluoro-7-nitro-1H-indol-3-yl)-3-[4-(trifluoromethyl)phenyl]propan-1-ol

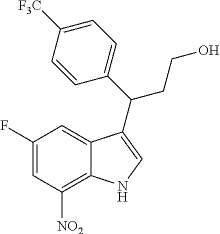

544 mg (1.28 mmol) of the compound from example 26A were initially charged in 10 ml of tetrahydrofuran, admixed at 0° C. with 55.9 mg (2.56 mmol) of lithium borohydride and stirred at RT overnight. Then a further 28.0 mg (1.28 mmol) of lithium borohydride were added and the reaction mixture was again stirred at RT overnight. Subsequently, water was added and extraction was effected with ethyl acetate. The organic phase was washed with water and saturated sodium chloride solution, dried over magnesium sulfate, filtered and concentrated. The residue was purified by means of flash chromatography (eluent: dichloromethane/methanol 100:1) to obtain 194 mg (40% of theory) of the title compound.

¹H NMR (400 MHz, DMSO-d₆): δ=2.13-2.24 (m, 1H), 2.29-2.39 (m, 1H), 3.30-3.42 (m, 2H), 4.50-4.56 (m, 2H), 6.59-7.65 (m, 5H), 7.84 (dd, 1H), 7.90 (dd, 1H), 11.9 (s, 1H).

LC-MS (method 9): Rₜ=2.70 min; MS (ESIpos): m/z=383 [M+H]⁺.

### Example 52

### N-(3-{3-Cyano-1-[4-(trifluoromethyl)phenyl]propyl}-5-fluoro-1H-indol-7-yl)methanesulfonamide

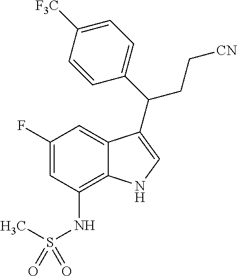

176 mg (460 μmol) of the compound from example 51 were initially charged in 10 ml of ethanol, admixed with 17.5 mg of palladium on carbon (10%) and hydrogenated at standard pressure and RT overnight. Then the mixture was filtered through Celite and washed through with ethanol, and the filtrate was concentrated to obtain 160 mg of the crude intermediate. 153 mg (433 μmol) of this intermediate were initially charged in dichloromethane under argon, admixed with 5.3 mg (43 μmol) of 4-N,N-dimethylaminopyridine, 149 mg (1.47 mmol) of triethylamine and 149 mg (1.30 mmol) of methanesulfonyl chloride, and stirred at RT overnight. Subsequently, ethyl acetate was added, the mixture was washed successively with 1 M hydrochloric acid, water and saturated aqueous sodium chloride solution, and the organic phase was dried over magnesium sulfate, filtered and concentrated. The residue was purified by means of preparative HPLC (RP18 column; eluent: acetonitrile/water gradient) to obtain 207 mg of the second intermediate. 149 mg of this intermediate were initially charged in 5 ml of dimethylformamide, admixed with 33.1 mg (508 μmol) of potassium cyanide and stirred at 80° C. overnight. The reaction mixture was then purified directly by means of preparative HPLC (RP18 column; eluent: acetonitrile/water gradient with addition of 1% formic acid) to obtain 63 mg of the title compound.

¹H NMR (400 MHz, DMSO-d₆): δ=2.29-2.38 (m, 1H), 2.39-2.45 (m, 2H), 2.46-2.55 (m, 1H), 3.03 (s, 3H), 4.31 (t, 1H), 6.89 (dd, 1H), 7.06 (dd, 1H), 7.53 (d, 1H), 7.58-7.67 (m, 4H), 9.56 (s, 1H), 10.9 (s, 1H).

MS (CIpos): m/z=457 [M+NH₄]⁺.

The enantiomers were separated by preparative HPLC on a chiral phase [column: Daicel Chiralpak AD-H, 5 μm 250 mm×20 mm, eluent: ethanol; flow rate: 10 ml/min; temperature: 40° C.; UV detection: 220 nm]:

### Enantiomer 52-1:

Rₜ=4.80 min [column: Daicel Chiralpak AD-H, 5 μm 250 mm×4.6 mm, eluent: ethanol; flow rate: 0.8 ml/min; temperature: 45° C.; UV detection: 220 nm];

### Enantiomer 52-2:

Rₜ=8.37 min [column: Daicel Chiralpak AD-H, 5 μm 250 mm×4.6 mm, eluent: ethanol; flow rate: 0.8 ml/min; temperature: 45° C.; UV detection: 220 nm].

### Example 53

### N-(3-{3-Cyano-1-[4-(trifluoromethyl)phenyl]propyl}-1H-indol-7-yl)ethanesulfonamide

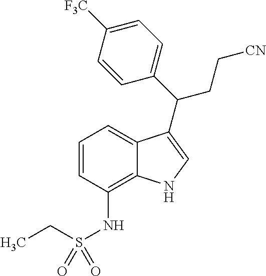

80.0 mg (239 μmol) of the compound from example 15 were initially charged in 1.5 ml of tetrahydrofuran and admixed at 0° C. with 48 μl (598 μmol) of pyridine and 57 μl (598 μmol) of ethanesulfonyl chloride. The reaction mixture was stirred at RT overnight and then at 50° C. for 4 h. A further 23 μl (239 μmol) of ethanesulfonyl chloride were added and 19 μl (239 μmol) of pyridine were added and the mixture was stirred again at RT overnight. Subsequently, another 35 μl (359 μmol) of ethanesulfonyl chloride and 29 μl (359 μmol) of pyridine were added and the mixture was stirred again at RT overnight. The last procedure was repeated once more. Finally, ethyl acetate was added, and the mixture was extracted successively with 1 M hydrochloric acid, water and saturated aqueous sodium chloride solution. The organic phase was dried over magnesium sulfate, filtered and concentrated. The residue was purified by means of preparative HPLC (RP18 column; eluent: acetonitrile/water gradient with addition of 0.1% formic acid) to obtain 48.7 mg of the intermediate. 45 mg of this intermediate were initially charged in 1 ml of di-methylformamide, admixed with 11.3 mg (174 μmol) of potassium cyanide and stirred at 80° C. for 3 h. Subsequently, ethyl acetate was added, the mixture was extracted with water and saturated sodium hydrogencarbonate solution, and the organic phase was dried over magnesium sulfate, filtered and concentrated. The crude product was purified by means of preparative HPLC (RP18 column; eluent: acetonitrile/water gradient with addition of 1% formic acid) to obtain 30.4 mg of the title compound.

¹H NMR (400 MHz, DMSO-d₆): δ=1.19 (t, 3H), 2.31-2.39 (m, 1H), 2.40-2.46 (m, 2H), 2.46-2.52 (m, 1H), 3.07 (q, 2H), 4.34 (t, 1H), 6.90 (t, 1H), 7.03 (d, 1H), 7.25 (d, 1H), 7.43-7.45 (m, 1H), 7.56-7.61 (m, 2H), 7.62-7.67 (m, 2H), 9.33 (s, 1H), 10.7 (s, 1H).

MS (ESIneg): m/z=434 [M−H]⁻

The enantiomers were separated by preparative HPLC on a chiral phase [column: chiral silica gel phase based on the selector poly(N-methacryloyl-L-leucine-D-menthylamide), 5 μm, 250 mm×30 mm; eluent: ethyl acetate; flow rate: 50 ml/min; temperature: 24° C.; UV detection: 260 nm]:

### Enantiomer 53-1:

Rₜ=3.68 min [column: chiral silica gel phase based on the selector poly(N-methacryloyl-L-leucine-D-menthylamide), 5 μm, 250 mm×4.6 mm; eluent: ethyl acetate; flow rate: 2 ml/min; temperature: 24° C.; UV detection: 260 nm];

### Enantiomer 53-2:

Rₜ=4.84 min [column: chiral silica gel phase based on the selector poly(N-methacryloyl-L-leucine-D-menthylamide), 5 μm, 250 mm×4.6 mm; eluent: ethyl acetate; flow rate: 2 ml/min; temperature: 24° C.; UV detection: 260 nm].

### B. EVALUATION OF THE PHARMACOLOGICAL ACTIVITY

### Abbreviations:

### DMEM Dulbecco's Modified Eagle Medium

DNA deoxyribonucleic acid
FCS fetal calf serum
HEPES 4-(2-hydroxyethyl)-1-piperazinylethanesulfonic acid

### PCR Polymerase Chain Reaction

Tris tris(hydroxymethyl)methylamine

The advantageous pharmacological properties of the inventive compounds can be demonstrated in the following assays:

1. Cellular In Vitro Assay to Determine the Inhibitory MR Activity and MR Selectivity Compared with Other Steroid Hormone Receptors

Antagonists of the human mineralocorticoid receptor (MR) are identified, and the efficacy of the compounds described herein is quantified with the aid of a recombinant cell line. The cell is originally derived from a hamster ovary epithelial cell (Chinese Hamster Ovary, CHO K1, ATCC: American Type Culture Collection, VA 20108, USA).

An established chimera system in which the ligand-binding domains of human steroid hormone receptors are fused to the DNA-binding domain of the yeast transcription factor GAL4 is used in this CHO K1 cell line. The GAL4-steroid hormone receptor chimeras produced in this way are cotransfected and stably expressed with a reporter construct in the CHO cells.

### Cloning:

To generate the GAL4-steroid hormone receptor chimeras, the GAL4 DNA-binding domain (amino acids 1-147) from the vector pFC2-dbd (from Stratagene) is cloned with the PCR-amplified ligand-binding domains of the mineralocorticoid receptor (MR, amino acids 734-985), of the glucocorticoid receptor (GR, amino acids 443-777), of the progesterone receptor (PR, amino acids 680-933) and of the androgen receptor (AR, amino acids 667-919) into the vector pIRES2 (from Clontech). The reporter construct, which contains five copies of the GAL4 binding site upstream of a thymidine kinase promoter, leads to expression of firefly luciferase (Photinus pyralis) after activation and binding of the GAL4-steroid hormone receptor chimeras by the respective specific agonists aldosterone (MR), dexamethasone (GR), progesterone (PR) and dihydrotestosterone (AR).

### Assay Procedure:

The MR, GR, PR and AR cells are plated out in medium (Optimem, 2.5% FCS, 2 mM glutamine, 10 mM HEPES) in 96-well (or 384- or 1536-well) microtiter plates the day before the assay, and are kept in a cell incubator (96% air humidity, 5% v/v CO₂, 37° C.). On the day of the assay, the substances to be tested are taken up in the abovementioned medium and added to the cells. About 10 to 30 minutes after addition of the test substances, the respective specific agonists of the steroid hormone receptors are added. After a further incubation time of 5 to 6 hours, the luciferase activity is measured with the aid of a video camera. The relative light units measured as a function of the substance concentration give a sigmoidal stimulation curve. The IC₅₀ values are calculated with the aid of the computer program GraphPad PRISM (Version 3.02).

Table A shows the IC₅₀ values of representative example compounds:

TABLE A

| | MR | GR | AR | PR |
|---|---|---|---|---|
| Example No. | IC₅₀ [μM] | IC₅₀ [μM] | IC₅₀ [μM] | IC₅₀ [μM] |
| 3-1 | 0.42 | 6.47 | 3.65 | 5.87 |
| 4-1 | 0.14 | 2.34 | 2.00 | 1.74 |
| 8-1 | 0.20 | 5.10 | 1.53 | 2.71 |
| 16 | 0.33 | 0.70 | 10 | 10 |
| 17-1 | 0.02 | 2.21 | 2.27 | 4.50 |
| 34 | 0.09 | 0.39 | 4.20 | 3.35 |

### 2. In Vivo Assay for Detection of the Cardiovascular Effect: Diuresis Studies on Conscious Rats in Metabolism Cages

Wistar rats (body weight 250-350 g) are kept with free access to feed (Altromin) and drinking water. From approx. 72 hours before the start of the test, the animals receive, instead of the normal feed, exclusively reduced-salt feed with a sodium chloride content of 0.02% (ssniff R/M−H, 10 mm with 0.02% Na, 50602-E081, ssniff Spezialdiäten GmbH, D-59494 Soest). During the test, the animals are housed singly in metabolism cages suitable for rats of this weight class (from Tecniplast Deutschland GmbH, D-82383 Hohenpeissenberg) with free access to reduced-salt feed and drinking water for about 24 hours. At the start of the test, the substance to be tested is administered into the animals' stomachs by means of gavage in a volume of 0.5 ml/kg of body weight of a suitable solvent. Control animals receive only solvent. Controls and substance tests are carried out in parallel on the same day. Control groups and substance-dose groups each consist of 6 to 8 animals. During the test, the urine excreted by the animals is continuously collected in a receiver on the base of the cage. The urine volume per unit time is determined separately for each animal, and the concentration of the sodium and potassium ions excreted in the urine is measured by standard methods of flame photometry. The sodium/potassium ratio is calculated from the measurements as a measure of the effect of the substance. The measurement intervals are typically the period up to 8 hours after the start of the test (day interval) and the period from 8 to 24 hours after the start of the test (night interval). In a modified test design, the urine is collected and measured at intervals of two hours during the day interval. In order to obtain a sufficient amount of urine for this purpose, the animals receive a defined amount of water by gavage at the start of the test and then at intervals of two hours.

### 3. DOCA/Salt Model

Administration of deoxycorticosterone acetate (DOCA) in combination with a high-salt diet and unilateral kidney removal in rats induces hypertension which is characterized by relatively low renin levels. A consequence of this endocrine hypertension (DOCA is a direct precursor of aldosterone) is, depending on the chosen DOCA concentration, cardiac hypertrophy and further end organ damage, for example to the kidney, which is characterized by proteinuria and glomerulosclerosis inter alia. It is thus possible in this rat model to investigate test substances for the presence of an antihypertrophic and end organ-protecting effect.

Male Sprague-Dawley (SD) rats of about 8 weeks in age (body weight between 250 and 300 grams) undergo left uninephrectomy. For this purpose, the rats are anesthetized with 1.5-2% isoflurane in a mixture of 66% N₂O and 33% O₂, and the kidney is removed through a flank incision. “Sham-operated” animals from which no kidney is removed serve later as control animals.

Uninephrectomized SD rats receive 1% sodium chloride in the drinking water and a subcutaneous injection of deoxycorticosterone acetate (dissolved in sesame oil; from Sigma) injected between the shoulder blades once a week (high dose: 100 mg/kg/week s.c.; normal dose: 30 mg/kg/week s.c.).

The substances which are to be studied for their protective effect in vivo are administered by gavage or via the feed (from Ssniff). One day before the start of the test, the animals are randomized and assigned to groups with an identical number of animals, usually n=10. Throughout the test, drinking water and feed are available ad libitum to the animals. The substances are administered via the feed or once a day by gavage for 4-8 weeks Animals serving as placebo group are treated in the same way but receive either only the solvent or the feed without test substance.

The effect of the test substances is determined by measuring hemodynamic parameters [blood pressure, heart rate, inotropism (dp/dt), relaxation time (tau), maximum left ventricular pressure, left-ventricular end-diastolic pressure (LVEDP)], by determining the weight of the heart, kidney and lung, by measuring the protein excretion, and by measuring gene expression of biomarkers (e.g. ANP, atrial natriuretic peptide, and BNP, brain natriuretic peptide) by means of RT/TaqMan PCR after RNA isolation from cardiac tissue.

Statistical analysis is effected by Student's t test after prior checking of the variances for homogeneity.

### 4. In Vivo Assay for Detecting Anti-Mineralocorticoid Activity on Conscious Dogs

The primary aim of the test is to study the influence of test compounds having antimineralocorticoid receptor activity on aldosterone-induced sodium retention. The procedure here is analogous to a published method: Rosenthale, M. E., Schneider F., Kassarich, J. & Datko, L. (1965): Determination of antialdosterone activity in normal dogs, Proc. Soc. Exp. Biol. Med., 118, 806-809.

Male or female beagles with a weight between 8 and 20 kilograms receive a standard diet and have free access to drinking water. On the days of the experiments, the dogs are fasting. Brief anesthesia is induced with Propofol (4-6 mg/kg intravenously; Propofol 1% Parke-Davis®, Godecke, Germany) in order to obtain an aliquot of urine (as starting value, day 1) with a bladder catheter.

On day 2, all the dogs receive, at about 16:00 hours, 0.3 mg of astonin, a metabolically stable aldosterone derivative (Astonin H, Merck, Germany).

The next morning (day 3), the test substance is administered to the dogs orally in a gelatin capsule. 5 hours later, blood is taken from the dogs to determine the plasma concentration of the substance. Subsequently, again in brief anesthesia, urine is obtained through a bladder catheter.

Treatment with the test substances leads, after 5 hours, to an increase in the sodium/potassium ratio in the urine (sodium and potassium determined by flame photometry). The positive control used is spironolactone, which likewise increases the sodium/potassium ratio in the urine in a dose-dependent manner; the negative control used is treatment with an empty capsule.

Evaluation is effected by comparing the sodium/potassium ratio in the urine between days 1 and 3. Alternatively, the sodium/potassium ratio can also be compared between placebo and substance on day 3.

### 5. Chronic Myocardial Infarction Model in Concious Rats

Male Wistar rats (body weight 280-300 g; Harlan-Winkelmann) are anesthetized with 5% isoflurane in an anesthesia cage, intubated, connected to a ventilation pump (ugo basile 7025 rodent, 50 strokes/min, 7 ml) and ventilated with 2% isoflurane/N₂O/O₂. The body temperature is maintained at 37-38° C. by a heating mat. 0.05 mg/kg Temgesic is given subcutaneously as analgesic. The chest is opened laterally between the third and fourth ribs, and the heart is exposed. The coronary artery of the left ventricle (LAD) is permanently ligated with an occlusion thread (Prolene 1 metric 5-0 Ethicon1H) passed underneath shortly below its origin (below the left atrium). The occurrence of myocardial infarction is monitored by an ECG measurement (Cardioline, Remco, Italy). The thorax is reclosed and the muscle layers are sutured with Ethibond excel 1 metric 5/0 6951H, and the epidermis is sutured with Ethibond excel 3/0 6558H. The surgical suture is wetted with a spray dressing (e.g. Nebacetin®N spray dressing, active ingredient: neomycin sulfate) and then the anesthesia is terminated.

One week after the LAD occlusion, the size of the myocardial infarction is estimated by echocardiography (Sequoia 512, Acuson). The animals are randomized and divided into individual treatment groups and a control group with no substance treatment. A further control included is a sham group in which only the surgical procedure, but not the LAD occlusion, was performed.

Substance treatment takes place over 8 weeks by gavage or by adding the test compound to the feed or drinking water. The animals are weighed weekly, and the water and feed consumption is determined every 14 days.

After treatment for 8 weeks, the animals are again anesthetized (2% isoflurane/N₂O/air) and a pressure catheter (Millar SPR-320 2F) is inserted via the carotid artery into the left ventricle. The heart rate, left ventricular pressure (LVP), left-ventricular end-diastolic pressure (LVEDP), contractility (dp/dt) and relaxation rate (τ) are measured there and analyzed with the aid of the Powerlab system (AD Instruments, ADI-PWLB-4SP) and the Chart 5 software (SN 425-0586). A blood sample is then taken to determine the plasma levels of the substance and plasma biomarkers, and the animals are sacrificed. The heart (heart chambers, left ventricle with septum, right ventricle), liver, lung and kidney are removed and weighed.

### 6. Stroke-Prone Spontaneously Hypertensive Rat Model

Administration of sodium chloride to the so-called stroke-prone spontaneously hypertensive rat (SP-SHR) leads in this model, paradoxically, to suspension of the physiological salt-induced repression of renin and angiotensin release after a few days. Thus, the hypertension in the SP-SHR animals is characterized by a relatively high renin level. Consequences of the developing hypertension are pronounced end-organ damage to the heart and the kidney, which is characterized by proteinuria and glomerulosclerosis inter alia, and general vascular changes. Thus, it is possible in particular for strokes to develop primarily through cerebrovascular lesions (“stroke-prone”), which lead to a high mortality of the untreated animals. It is thus possible in this rat model to study test substances for blood pressure-lowering and end organ-protecting effect.

One day before the start of the test, male SP-SH rats approximately 10 weeks of age (body weight between 190 and 220 g) are randomized and assigned to groups with an equal number of animals, usually n=12-14. Throughout the test, drinking water containing sodium chloride (2% NaCl) and feed are available ad libitum to the animals. The substances are administered once a day by gavage or with the feed (Ssniff, Germany) for 6-8 weeks Animals treated in the same way but receiving either only the solvent or the feed without test substance serve as placebo group. In the context of a mortality study, the test is terminated when about 50% of the placebo-treated animals have died.

The effect of the test substances is followed by measuring the changes in the systolic blood pressure (via a tail cuff) and the protein excretion in the urine. There are post mortem determinations of the weights of heart, kidney and lung, and histopathological analyses of the heart, kidney and brain with semiquantitative scoring of the histological changes. Various biomarkers (e.g. ANP, atrial natriuretic peptide, and BNP, brain natriuretic peptide, KIM-1, kidney-induced molecule 1, osteopontin-1) are determined by means of RT/TaqMan PCR following RNA isolation from cardiac and renal tissue or serum or plasma.

Statistical analysis is carried out using Student's t test after prior checking of the variances for homogeneity.

### C. WORKING EXAMPLES OF PHARMACEUTICAL COMPOSITIONS

The inventive compounds can be converted to pharmaceutical formulations as follows:

### Tablet:

### Composition:

100 mg of the inventive compound, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg, diameter 8 mm, radius of curvature 12 mm.

### Production:

The mixture of inventive compound, lactose and starch is granulated with a 5% solution (w/w) of the PVP in water. After drying, the granules are mixed with the magnesium stearate for 5 minutes. This mixture is pressed with a conventional tableting press (for tablet format see above). The guide value used for the pressing is a pressing force of 15 kN.

### Suspension for Oral Administration:

### Composition:

1000 mg of the inventive compound, 1000 mg of ethanol (96%), 400 mg of Rhodigel® (xanthan gum from FMC, Pennsylvania, USA) and 99 g of water.

A single dose of 100 mg of the inventive compound corresponds to 10 ml of oral suspension.

### Production:

The Rhodigel is suspended in ethanol and the inventive compound is added to the suspension. The water is added while stirring. The mixture is stirred for approx. 6 h until swelling of the Rhodigel has ended.

### Solution for Oral Administration:

### Composition:

500 mg of the inventive compound, 2.5 g of polysorbate and 97 g of polyethylene glycol 400.20 g of oral solution correspond to an individual dose of 100 mg of the inventive compound.

### Production:

The inventive compound is suspended in the mixture of polyethylene glycol and polysorbate while stirring. The stirring operation is continued until dissolution of the inventive compound is complete.

i.v. Solution:

The inventive compound is dissolved in a concentration below the saturation solubility in a physiologically acceptable solvent (e.g. isotonic saline, glucose solution 5% and/or PEG 400 solution 30%). The solution is subjected to sterile filtration and dispensed into sterile and pyrogen-free injection vessels.

1. A compound of the formula (I) 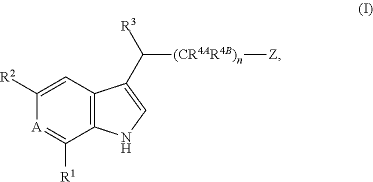
in which
A is C—R⁵ or N
where
R⁵ is hydrogen, fluorine, chlorine or (C₁-C₄)-alkyl,
R¹ is halogen, cyano, nitro, (C₁-C₆)-alkyl, (C₁-C₆)-alkoxy, amino, mono-(C₁-C₆)-alkylamino, di-(C₁-C₆)-alkylamino or a group of the formula —(CH₂)ₚ—NR⁶—SO₂—R⁷,
where (C₁-C₆)-alkyl and (C₁-C₆)-alkoxy may each be substituted by 1 to 3 fluorine substituents,
where (C₁-C₆)-alkyl and (C₁-C₆)-alkoxy may each be substituted by a substituent selected from the group of hydroxyl and (C₁-C₄)-alkoxy
and where
p is 0, 1 or 2,
R⁶ is hydrogen or (C₁-C₄)-alkyl,
and
R⁷ is (C₁-C₆)-alkyl, (C₃-C₇)-cycloalkyl, phenyl, benzyl or 5- or 6-membered heteroaryl,
in which phenyl, benzyl and 5- or 6-membered heteroaryl may each be substituted by 1 to 3 substituents selected independently from the group of halogen, cyano, nitro, (C₁-C₄)-alkyl, trifluoromethyl, hydroxyl, (C₁-C₄)-alkoxy, trifluoromethoxy and amino,
R² is hydrogen, fluorine, chlorine or (C₁-C₄)-alkyl,
R³ is phenyl or naphthyl,
where phenyl and naphthyl may each be substituted by 1 to 3 substituents selected independently from the group of halogen, cyano, nitro, (C₁-C₄)-alkyl, trifluoromethyl, (C₁-C₄)-alkoxy, trifluoromethoxy, mono-(C₁-C₄)-alkylamino, di-(C₁-C₄)-alkylamino, aminocarbonyl, mono-(C₁-C₄)-alkylaminocarbonyl and di-(C₁-C₄)-alkylaminocarbonyl,
n is 2 or 3,
R^{4A} is hydrogen, fluorine or (C₁-C₄)-alkyl,
R^{4B} is hydrogen, fluorine or (C₁-C₄)-alkyl,
and
Z is hydroxyl or cyano,
or a salt thereof.

2. The compound of formula (I) as claimed in claim 1, in which
A is C—R⁵
where
R⁵ is hydrogen,
R¹ is chlorine, bromine, cyano, nitro, (C₁-C₄)-alkyl, (C₁-C₄)-alkoxy, amino, mono-(C₁-C₄)-alkylamino, di-(C₁-C₄)-alkylamino or a group of the formula —(CH₂)ₚ—NR⁶—SO₂—R⁷,
where (C₁-C₄)-alkyl and (C₁-C₄)-alkoxy may each be substituted by 1 to 3 fluorine substituents,
where (C₁-C₄)-alkyl and (C₁-C₄)-alkoxy may each be substituted by one substituent selected from the group of hydroxyl and (C₁-C₄)-alkoxy,
and where
p is 0 or 1,
R⁶ is hydrogen or methyl,
and
R⁷ is (C₁-C₆)-alkyl, (C₃-C₆)-cycloalkyl, phenyl, benzyl or 5- or 6-membered heteroaryl,
in which phenyl, benzyl and 5- or 6-membered heteroaryl may each be substituted by 1 or 2 substituents selected independently from the group of fluorine, chlorine, bromine, cyano, nitro, (C₁-C₄)-alkyl, trifluoromethyl, hydroxyl, (C₁-C₄)-alkoxy, trifluoromethoxy and amino,
R² is hydrogen, fluorine or methyl,
R³ is phenyl or naphthyl,
where phenyl and naphthyl may each be substituted by 1 or 2 substituents selected independently from the group of fluorine, chlorine, bromine, cyano, nitro, (C₁-C₄)-alkyl, trifluoromethyl, (C₁-C₄)-alkoxy and trifluoromethoxy,
n is 2 or 3,
R^{4A} is hydrogen, fluorine or methyl,
R^{4B} is hydrogen, fluorine or methyl,
and
Z is hydroxyl or cyano,
or a salt thereof.

3. A compound of the formula (I) as claimed in claim 1, in which
A is C—R⁵
where
R⁵ is hydrogen,
R¹ is bromine, cyano, methyl, ethyl, trifluoromethyl or a group of the formula —(CH₂)ₚ—NR⁶—SO₂—R⁷,
and where
p is 0,
R⁶ is hydrogen,
and
R⁷ is methyl or ethyl,
R² is hydrogen or fluorine,
R³ is phenyl or naphthyl,
where phenyl may be substituted by 1 or 2 substituents selected independently from the group of fluorine, chlorine, methyl and trifluoromethyl,
n is 2 or 3,
R^{4A} is hydrogen,
R^{4B} is hydrogen,
and
Z is hydroxyl or cyano,
or a salt thereof.

4. A process for preparing compounds of the formula (I) as defined in claim 1, in which R^{4A} and R^{4B} are each hydrogen, characterized in that
[A] first an indole derivative of the formula (II) 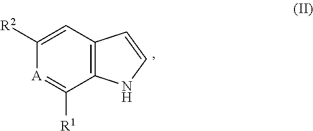
in which A, R¹ and R² are each as defined in claim 1,
in an inert solvent, optionally in the presence of an acid and/or base, is condensed with a benzaldehyde of the formula (III) 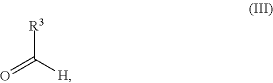
in which R³ is as defined in claim 1,
and a malonic ester of the formula (IV) 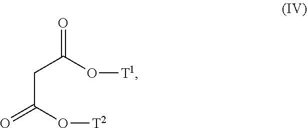
in which
T¹ and T² are the same or different and are each (C₁-C₄)-alkyl, or both together form a >C(CH₃)₂ bridge,
to give a compound of the formula (V) 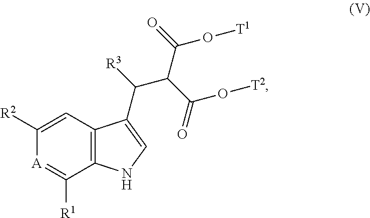
in which A, R¹, R², R³, T¹ and T² are each as defined in claim 1,
then the diester is cleaved with decarboxylation to give a compound of the formula (VI) 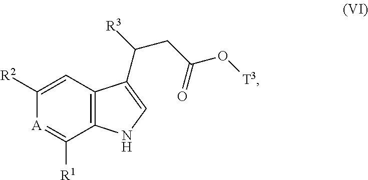
in which A, R¹, R² and R³ are each as defined in claim 1
and
T³ is hydrogen or (C₁-C₄)-alkyl,
and the latter compound is then converted in an inert solvent, using a suitable reducing agent, for example lithium aluminum hydride, to the inventive compound of the formula (I-1) 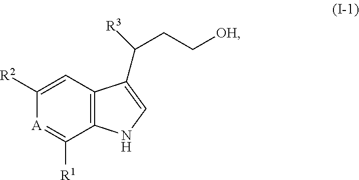
in which A, R¹, R² and R³ are each as defined in claim 1,
[B] the compound of the formula (I-1) is in turn reacted by standard methods, via a compound of the formula (VII) 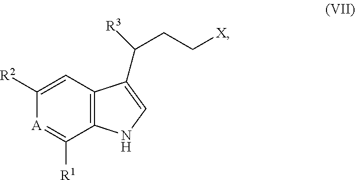
in which A, R¹, R² and R³ are each as defined in claim 1
and
X is a suitable leaving group, for example halogen, mesylate, tosylate or triflate,
and subsequent substitution reaction with an alkali metal cyanide to give the inventive compound of the formula (I-2) 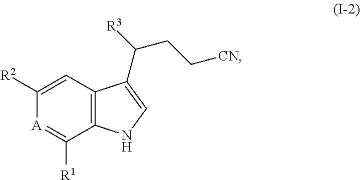
in which A, R¹, R² and R³ are each as defined in claim 1,
[C] the compound of the formula (I-2) is in turn first hydrolyzed to the carboxylic acid of the formula (VIII) 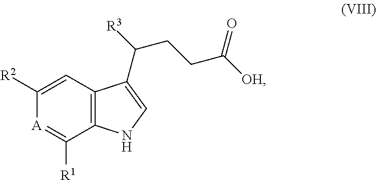
in which A, R¹, R² and R³ are each as defined in claim 1,
and the latter compound is then converted in an inert solvent, using a suitable reducing agent, for example lithium aluminum hydride, to the inventive compound of the formula (I-3) 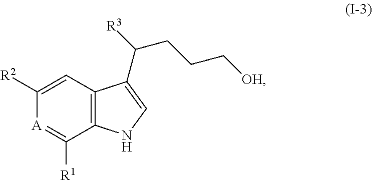
in which A, R¹, R² and R³ are each as defined in claim 1
and
[D] the compound of the formula (I-3) is in turn reacted by standard methods, via a compound of the formula (IX) 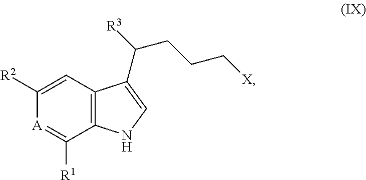
in which A, R¹, R² and R³ are each as defined in claim 1
and
X is a suitable leaving group, for example halogen, mesylate, tosylate or triflate,
and subsequent substitution reaction with an alkali metal cyanide to give the inventive compound of the formula (I-4) 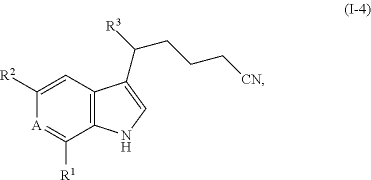
in which A, R¹, R² and R³ are each as defined in claim 1,
and the resulting compounds of the formula (I-1), (I-2), (I-3) or (I-4) are optionally separated by methods known to those skilled in the art into the enantiomers and/or diastereomers thereof and/or converted using the appropriate (i) solvents and/or (ii) bases or salts thereof.

5. (canceled)

6. (canceled)

7. (canceled)

8. A pharmaceutical composition comprising the compound of claim 1 and an inert, nontoxic, pharmaceutically suitable excipient.

9. The pharmaceutical composition of claim 8, further comprising at least one additional active ingredient selected from the group consisting of an ACE inhibitor, a renin inhibitor, an angiotension II receptor antagonist, a beta blocker, acetylsalicylic acid, a diuretic, a calcium antagonist, a statin, a digitalis (digoxin) derivative, a vasopressin antagonist, an adenosine A1 antagonist, a calcium sensitizer, a nitrate, and a antithrombotic.

10. (canceled)

11. A method for treatment and/or prophylaxis of aldosteronism, high blood pressure, acute and chronic heart failure, the consequences of heart failure, liver cirrhosis, kidney failure and stroke by administering to a patient in need thereof effective amount of at least one compound of claim 1.
